(19) United States
(12) Patent Application Publication
(10) Pub. No.: US 2003/0177534 A1
(43) Pub. Date: Sep. 18, 2003

(54) Plant glutamine:fructose-6-phosphate amidotransferase nucleic acids

(75) Inventors: Scott E. Nichols, Westchester, PA (US); Tie Wang, Des Moines, IA (US); Patricia Lynne Fallis, Polk City, IA (US); Kanwarpal S. Dhugga, Johnston, IA (US)

Correspondence Address: PIONEER HI-BRED INTERNATIONAL INC., 7100 N.W. 62ND AVENUE, P.O. BOX 1000, JOHNSTON, IA, 50131, US

(73) Assignee: Pioneer Hi-Bred International, Inc.

(21) Appl. No.: 10/323,020

(22) Filed: Dec. 17, 2002

Related U.S. Application Data
(63) Continuation of application No. 09/379,779, filed on Aug. 24, 1999, now abandoned.
(60) Provisional application No. 60/097,881, filed on Aug. 25, 1998.

(51) Int. Cl. A01H 5/00; C12N 9/10; A01H 1/00; C12N 5/04; C12N 15/82

(52) U.S. Cl. 800/284; 800/320.1; 435/193; 435/412

(57) ABSTRACT

Isolated nucleic acid molecules are provided that encode maize glutamine:fructose-6-phosphate amidotransferase (GFAT) and variant GFAT proteins. These nucleic acid molecules can be used to produce transgenic plants that synthesize cationic starch. Also provided are vectors capable of expressing such nucleic acid molecules, host cells containing such vectors, and polypeptides encoded by such nucleic acids.

### CROSS-REFERENCE TO RELATED APPLICATIONS

This application is a continuation of co-pending U.S. patent application Ser. No. 09/379,779 filed Aug. 24, 1999, which application claims the benefit of U.S. Patent Application No. 60/097,881 filed Aug. 25, 1998, now abandoned, which is herein incorporated by reference.

### TECHNICAL FIELD

The present invention relates generally to methods of regulating the starch composition of plants. In particular, the present invention relates to a novel glutamine:fructose-6-phosphate amidotransferase (GFAT) nucleic acid, variant forms of the nucleic acid, and the use of such GFAT nucleic acids to produce plants that synthesize cationic starch.

### BACKGROUND OF THE INVENTION

Due to its unique physical properties, starch is not only used in food products, but has a variety of industrial applications, including paper production, textiles, adhesives, flocculants, and building materials (see, for example, Kirby, “Non-Food Uses of Starch,” in Developments in Carbohydrate Chemistry (Alexander and Zobel, eds.), pages 371-386 (The American Association of Cereal Chemists 1992); Watson, “Corn Marketing, Processing, and Utilization,” in Corn and Corn Improvement (Sprague and Dudley, eds.), pages 881-940 (American Society of Agronomy, Inc. et al., 1988)). Plant starch for non-food use is obtained mainly from maize, potato, tapioca, and wheat. Barley, rice, and sago palm are considered as secondary sources of starch. In the United States, corn provides over 95% of the raw material for starch.

Regardless of its source, starch is comprised of α-D-glucose units. Amylose, which comprises about 27% of the corn starch granule, is a linear polysaccharide composed of 1,000 to 10,000 glucose residues connected by α-1,4-glucosidic residues. Amylopectin, 73% of the granule, is comprised of short α-1,4-linked chains connected by α-1,6-glucosyl branching linkages. Differences in starch structure arise from varying amounts of amylose and amylopectin, the frequency of branching in amylopectin, and the length of amylose and amylopectin chains. For example, the starch of waxy maize contains only the branched chain amylopectin, while the starch of the ae maize mutant, designated as “amylomaize or high-amylose corn starch,” contains 55-60% amylose.

The versatility of starch has been enhanced by chemically modifying a natural product to obtain derivatives with new properties. One significant derivative is cationic starch that is used in a variety of industrial processes, including textile sizing, adhesives, water purification, detergents and paper manufacture. In paper production, for example, cationic starch provides adhesive to bond the anionic wood fibers, increases drainage, and eases the drying of paper sheets.

Commercially available cationic starches are typically amino alkyl or quaternary derivatives of corn, potato, tapioca or waxy starch (Kirby; Watson; Mentzer, “Starch in the Paper Industry,” in Starch: Chemistry and Technology (Whistler et al., eds.), pages 543-574 (Academic Press 1984)). For example, Dishburger et al., Canadian Patent No. 888,190 (1971), teach a method for preparing cationic starch in which starch is heated with polyalkylenimine or polyalkylenepolyamine. Moreover, Matsunaga et al., Japanese Patent Application No. JP 8594937 (1986), describe a method for producing cationic starch which requires heating corn starch with a solution of 3-chloro-2-hydroxypropyltrimethylammonium chloride solution.

It would be preferable to avoid these chemical modification processes and obtain cationic starch directly from plants, such as maize. A pathway for the biosynthesis of cationic starch, however, is normally not found in maize or any other starch-storing plant tissue. Accordingly, a need exists for producing genetically engineered plants that have the capacity to synthesize cationic starch. The present invention discloses novel nucleic acids that can be used to obtain such plants.

### SUMMARY OF THE INVENTION

The present invention provides nucleic acid molecules that encode plant glutamine:fructose-6-phosphate amidotransferase (GFAT), as well as modified GFAT proteins. More specifically, one aspect of the present invention provides isolated nucleic acid molecules encoding a maize GFAT.

The present invention provides an isolated nucleic acid molecule comprising a nucleotide sequence selected from the group consisting of:

(a) a polynucleotide that encodes a plant glutamine:fructose-6-phosphate amidotransferase protein;

(b) a polynucleotide that encodes the polypeptide of SEQ ID NO: 2;

(c) a polynucleotide having at least 80% sequence identity to the sequence of SEQ ID NO: 1, wherein the % identity is determined by Wisconsin Package Version 9.1, GapWeight 5 and GapLengthWeight 1, Genetics Computer Group (GCG) Madison, Wis.;

(d) a polynucleotide that hybridizes under stringent conditions to a nucleic acid molecule having the nucleotide sequence of SEQ ID NO: 1, wherein the conditions include hybridization in 50% formamide, 1 M NaCl, 1% SDS at 37° C., and a wash in 0.1×SSC at 60° C.;

(e) a polynucleotide comprising at least 45 contiguous nucleotides SEQ ID NO: 1;

(f) a polynucleotide which is complementary to (a), (b), (c), (d), or (e); and

(g) a nucleic acid molecule that encodes a functional fragment of the polypeptide encoded by (a), (b), (c) or (d).

Also provided by the present invention are vectors comprising such nucleic acids, host cells, and plants that contain these vectors.

Also provided is an isolated polypeptide comprising a member selected from the group consisting of:

(a) a plant glutamine:fructose-6-phosphate amidotransferase protein;

(b) a polypeptide encoded by a member of claim 1;

(c) an amino acid sequence having at least 80% identity to the amino acid sequence of SEQ ID NO: 2, wherein the % identity is determined by Wisconsin Package Version 9.1, GapWeight 12 and GapLengthWeight 4, Genetics Computer Group (GCG) Madison, Wis.;

(d) a polypeptide having the sequence of SEQ ID NO: 2;

(e) a polypeptide of at least 55 contiguous amino acids encoded by the isolated nucleic acid of SEQ ID NO: 2; and

(f) a functional fragment of (a), (b), (c), or (d).

Other aspects of the present invention include methods for producing a plant that expresses GFAT comprising the steps of:

(a) stably transforming a plant cell with a glutamine:fructose-6-phosphate amidotransferase polynucleotide;

(b) regenerating a plant from the recombinant plant cell, wherein the plant expresses the GFAT protein encoded by the expression vector.

Other aspects of the invention include a method for producing cationic starch in plants comprising:

(a) stably transforming a plant cell with one or more polynucleotides encoding enzymes selected from the group consisting of glutamine:fructose-6-phosphate amidotransferase, UDP glucose pyrophosphorylase, phosphoglucomutase; starch synthase and glycogen synthase, wherein the polynucleotide is operably linked to a promoter capable of driving expression in plants; and

(b) regenerating a plant from the recombinant plant cell, wherein the plant produces cationic starch.

The present invention also provides methods of producing a plant that produces cationic starch comprising 2-amino anhydroglucose moieties, and transgenic plants and plant cells that contain the nucleic acid molecules, or vectors, described herein. The present invention further includes cationic starch produced by transgenic plant cells.

These and other aspects of the present invention will become evident upon reference to the following detailed description and attached drawings. In addition, various references are identified below and are incorporated by reference in their entirety.

### DETAILED DESCRIPTION OF THE INVENTION

1. Definitions

In the description that follows, a number of terms are used extensively. The following definitions are provided to facilitate understanding of the invention.

A “structural gene” is a nucleotide sequence that is transcribed into messenger RNA (mRNA), which is then translated into a sequence of amino acids characteristic of a specific polypeptide.

As used herein, “nucleic acid” or “nucleic acid molecule” refers to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally-occurring nucleotides (such as deoxyribonucleotides and ribonucleotides), or analogs of naturally-occurring nucleotides (e.g., α-enantiomeric forms of naturally-occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocylcic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids,” which comprise naturally-occurring or modified nucleic acid bases attached to a polyamide backbone. Nucleic acids can be either single stranded or double stranded.

The term “isolated” refers to material, such as a nucleic acid molecule or a protein, which is: (1) substantially or essentially free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a locus in the cell (e.g., genome or subcellular organelle) not native to a material found in that environment. The alteration to yield the synthetic material can be performed on the material within or removed from its natural state. For example, a naturally occurring nucleic acid molecule becomes an isolated nucleic acid molecule if it is altered, or if it is transcribed from DNA which has been altered, by non-natural, synthetic (i.e., “man-made”) methods performed within the cell from which it originates (See, e.g., Kmiec, U.S. Pat. No. 5,565,350; Zarling et al., PCT/US93/03868). Likewise, a naturally occurring nucleic acid (e.g., a promoter) becomes “isolated” if it is introduced by non-naturally occurring means to a locus of the genome not native to that nucleic acid. Nucleic acids molecules which are “isolated” as defined herein, are also referred to as “heterologous” nucleic acid molecules.

As used herein, a “glutamine:fructose-6-phosphate amidotransferase nucleic acid” (GFAT nucleic acid) is a nucleic acid molecule encoding glutamine:fructose-6-phosphate amidotransferase, a protein that catalyzes the rate-limiting step of the hexosamine biosynthetic pathway. Specifically, a GFAT enzyme catalyzes the formation of glucosamine-6-phosphate and glutamate from fructose-6-phosphate and glutamine. The amino acid sequence of a representative form of maize GFAT has been deduced and is presented in SEQ ID NO: 2.

Within the context of this invention, a “GFAT variant or functional fragment” refers to a nucleic acid molecule that encodes a polypeptide having an amino acid sequence that is a modification of SEQ ID NO: 2. Such variants include naturally-occurring polymorphisms of maize GFAT nucleic acids, as well as synthetic nucleic acids that contain conservative amino acid substitutions of the amino acid sequence of SEQ ID NO: 2. Additional forms of GFAT variants are nucleic acid molecules that contain insertions or deletions of the maize GFAT encoding sequences described herein. Preferred variant GFAT nucleic acids include plant GFAT nucleic acids.

As used herein, two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs, and published methods for determining optimal alignment between two nucleotide or amino acid sequences and comparing the aligned sequences (see, for example, Peruski and Peruski, The Internet and the New Biology: Tools for Genomic and Molecular Research (ASM Press, Inc. 1997), Wu et al (eds.), “Information Superhighway and Computer Databases of Nucleic Acids and Proteins,” in Methods in Gene Biotechnology, pages 123-151 (CRC Press, Inc. 1997), and Bishop (ed.), Guide to Human Genome Computing, 2^{nd} Edition (Academic Press, Inc. 1998)). As an illustration, nucleotide sequences can be compared using the BLASTN program (National Center for Biotechnology Information) with default parameters. For purposes of defining the invention, Wisconsin Package Version 9.1, GapWeight 12 and GapLengthWeight 4, Genetics Computer Group (GCG) Madison, Wis.

GFAT variants should preferably have at least an 80% amino acid sequence identity to SEQ ID NO: 2, and within certain embodiments, greater than 85%, 90%, 92%, 94%, 96%, or 98% identity. Alternatively, GFAT variants can be identified by having at least a 70% nucleotide sequence identity to SEQ ID NO: 1. Moreover, the present invention contemplates GFAT variants having greater than 75%, 80%, 85%, 90%, or 95% identity to SEQ ID NO: 1.

Nucleic acid molecules encoding a GFAT variant protein can also be identified by determining whether the molecule hybridize under stringent conditions with a reference nucleic acid molecule having a portion of the nucleotide sequence of SEQ ID NO: 1. Reference nucleic acid molecules may contain 10 to 50 nucleotides, between 50 to 500 nucleotides, between 500 to 1000 nucleotides, or greater than 1000 nucleotides.

The terms “stringent conditions” or “stringent hybridization conditions” includes reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide.

Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60° C. The time for hybridization is not critical and will generally be from 4-16 hours.

Specificity is typically the function of post-hybridization washes, the critical factors being the ionic strength and temperature of the final wash solution. For DNA-DNA hybrids, the Tm can be approximated from the equation of Meinkoth and Wahl, Anal. Biochem. 138:267 (1984): Tm=81.5° C.+16.6 (log M)+0.41 (%GC)−0.61 (% form)−500/L; where M is the molarity of monovalent cations, %GC is the percentage of guanosine and cytosine nucleotides in the DNA, % form is the percentage of formamide in the hybridization solution, and L is the length of the hybrid in base pairs. The Tm is the temperature (under defined ionic strength and pH) at which 50% of a complementary target sequence hybridizes to a perfectly matched probe. Tm is reduced by about 1° C. for each 1% of mismatching; thus, Tm, hybridization and/or wash conditions can be adjusted to hybridize to sequences of the desired identity. For example, if sequences with >90% identity are sought, the Tm can be decreased 10° C.

Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. If the desired degree of mismatching results in a Tm of less than 45° C. (aqueous solution) or 32° C. (formamide solution) it is preferred to increase the SSC concentration so that a higher temperature can be used. An extensive guide to the hybridization of nucleic acids is found in Tijssen, Laboratory Techniques in Biochemistry and Molecular Biology—Hybridization with Nucleic Acid Probes, Part I, Chapter 2 “Overview of principles of hybridization and the strategy of nucleic acid probe assays,” (Elsevier 1993), and by Ausubel, et al. (Eds.), Current Protocols in Molecular Biology, Chapter 2, (Greene Publishing and Wiley-Interscience 1995).

Regardless of the particular nucleotide sequence of a variant GFAT nucleic acid, the nucleic acid encodes an enzyme that catalyzes the typical reaction described above. More specifically, variant GFAT nucleic acids encode enzymes which exhibit at least 50%, and preferably, greater than 70, 80 or 90%, of the activity of the enzyme having the amino acid sequence of SEQ ID NO: 2, as determined by an assay described herein.

Within the context of this invention, a “functional fragment” of a GFAT nucleic acid refers to a nucleic acid molecule that encodes a portion of a GFAT polypeptide which possesses GFAT enzymatic activity. Similarly, a “functional fragment” of GFAT is a polypeptide exhibiting GFAT activity. For example, a functional fragment of the maize GFAT nucleic acid described herein comprises a portion of the nucleotide sequence of SEQ ID NO: 1, and encodes a polypeptide that can convert fructose-6-phosphate to glucosamine-6-phosphate.

A “promoter” is a nucleotide sequence that directs the transcription of a structural gene. Typically, a promoter is located in the 5′ region of a gene, proximal to the transcriptional start site of a structural gene. If a promoter is an inducible promoter, then the rate of transcription increases in response to an inducing agent. In contrast, the rate of transcription is not regulated by an inducing agent if the promoter is a constitutive promoter. A promoter contains essential nucleotide sequences for promoter function, including the TATA box and start of transcription.

A “regulatory element” is a nucleotide sequence that modulates the activity of a promoter. For example, a regulatory element may contain a nucleotide sequence that binds with cellular factors enabling transcription exclusively or preferentially in particular cells, tissues, organelles, or plastids. These types of regulatory elements are normally associated with genes that are expressed in a “cell-specific,” “tissue-specific,” “organelle-specific,” or “plastid-specific” manner.

An “enhancer” is a type of regulatory element that can increase the efficiency of transcription, regardless of the distance or orientation of the enhancer relative to the start site of transcription.

A “transit peptide” refers to an amino acid sequence that directs the transport of a fused protein into a plant organelle or plastid. Such organelles and plastids include but are not limited to leucoplasts, amyloplasts, chloroplasts, or mitochondria.

A “fusion protein” is a hybrid protein expressed by a nucleic acid molecule comprising nucleotide sequences of at least two genes. In the context of the present invention, a fusion protein comprises GFAT amino acid sequences and additional amino acid sequences. For example, a fusion protein can comprise amino acid sequences of a transit peptide joined with an amino acid sequence of at least part of a GFAT enzyme. As another example, a fusion protein can comprise at least part of a GFAT sequence fused with a polypeptide that binds an affinity matrix. Such fusion proteins are useful for isolating large quantities of GFAT protein using affinity chromatography.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand.

The term “expression” refers to the biosynthesis of a gene product. For example, in the case of a structural gene, expression involves transcription of the structural gene into mRNA and the translation of mRNA into one or more polypeptides. In contrast, the expression of a ribozyme gene, discussed below, results in the biosynthesis of a nucleic acid as the end product.

A “cloning vector” is a nucleic acid molecule, such as a plasmid, cosmid, or bacteriophage, that has the capability of replicating autonomously in a host cell. Cloning vectors typically contain one or a small number of restriction endonuclease recognition sites at which foreign nucleotide sequences can be inserted in a determinable fashion without loss of an essential biological function of the vector, as well as nucleotide sequences encoding a marker gene that is suitable for use in the identification and selection of cells transformed with the cloning vector. Examples of marker genes include genes that provide resistance to tetracycline, chloramphenicol, or ampicillin.

An “expression vector” is a nucleic acid molecule encoding a gene that is expressed in a host cell. Typically, gene expression is placed under the control of a promoter, and optionally, under the control of at least one regulatory element. Such a gene is said to be “operably linked to” the promoter. Similarly, a regulatory element and a promoter are operably linked if the regulatory element modulates the activity of the promoter. The product of a gene expressed by an expression vector is referred to as an “exogenous” gene product. For example, a maize cell comprising a vector that expresses a maize GFAT nucleic acid will contain mRNA of exogenous GFAT encoded by vector nucleotide sequences (i.e., this GFAT mRNA is encoded by an exogenous gene). Such a plant cell may also contain “endogenous” GFAT mRNA that is a transcript of genomic GFAT nucleotide sequences.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

“Cationic starch” is a polysaccharide that has a positive charge, and that is comprised of amylose and/or amylopectin. In the present context, cationic starch is also characterized by the presence of at least one 2-amino anhydroglucose moiety per starch molecule. Preferably, a molecule of cationic starch contains at least three 2-amino anhydroglucose moieties per 100 anhydroglucose moieties.

In eukaryotes, RNA polymerase II catalyzes the transcription of a structural gene to produce mRNA. A nucleic acid molecule can be designed to contain an RNA polymerase II template in which the RNA transcript has a sequence that is complementary to that of a specific mRNA. The RNA transcript is termed an “anti-sense RNA” and a nucleic acid molecule that encodes the anti-sense RNA is termed an “anti-sense gene.” Anti-sense RNA molecules are capable of binding to mRNA molecules, resulting in an inhibition of mRNA translation.

Similarly, an “anti-sense oligonucleotide specific for GFAT” or a “GFAT anti-sense oligonucleotide” is an oligonucleotide having a sequence (a) capable of forming a stable triplex with a portion of the GFAT nucleic acid, or (b) capable of forming a stable duplex with a portion of an mRNA transcript of the GFAT nucleic acid.

A “ribozyme” is a nucleic acid molecule that contains a catalytic center. The term includes RNA enzymes, self-splicing RNAs, self-cleaving RNAs, and nucleic acid molecules that perform these catalytic functions. A nucleic acid molecule that encodes a ribozyme is termed a “ribozyme gene.”

An “external guide sequence” is a nucleic acid molecule that directs the endogenous ribozyme, RNase P, to a particular species of intracellular mRNA, resulting in the cleavage of the mRNA by RNase P. A nucleic acid molecule that encodes an external guide sequence is termed an “external guide sequence gene.”

2. Isolation of GFAT Nucleic Acids

As described herein, DNA molecules encoding a maize GFAT nucleic acid have been isolated from a cDNA library. The nucleotide and predicted amino acid sequences of the maize GFAT nucleic acid are shown in SEQ ID NOS: 1 and 2, respectively. DNA molecules encoding this maize GFAT nucleic acid can be obtained by screening a maize cDNA or genomic library using polynucleotide probes based upon SEQ ID NO: 1. These techniques are standard and well-established.

For example, the first step in the preparation of a cDNA library is to isolate RNA from plant cells. Total RNA can be prepared from maize tissue using techniques well-known to those in the art. In general, RNA isolation techniques must provide a method for breaking plant cells, a means of inhibiting RNase-directed degradation of RNA, and a method of separating RNA from DNA, protein, and polysaccharide contaminants.

For example, total RNA can be isolated by freezing plant tissue in liquid nitrogen, grinding the frozen tissue with a mortar and pestle to lyse the cells, extracting the ground tissue with a solution of phenol/chloroform and aqueous buffer to remove proteins, and separating RNA from the remaining impurities by selective precipitation with lithium chloride (see, for example, Ausubel et al. (eds.), Current Protocols in Molecular Biology, pages 4.3.1-4.3.4 (Wiley Interscience 1990) [“Ausubel (1990)”]; Sharrock et al., Genes and Development 3:1745, 1989).

Alternatively, total RNA can be isolated from plant tissue by extracting ground tissue with guanidinium isothiocyanate, extracting with organic solvents, and separating RNA from contaminants using differential centrifugation (see, for example, Strommer et al., “Isolation and characterization of Plant mRNA,” in Methods in Plant Molecular Biology and Biotechnology, Glick et al. (eds.), pages 49-65 (CRC Press 1993)).

In order to construct a cDNA library, poly(A)⁺ RNA must be isolated from a total RNA preparation. Poly(A)⁺ RNA can be isolated from total RNA by using the standard technique of oligo(dT)-cellulose chromatography (see, for example, Strommer et al., supra.).

Double-stranded cDNA molecules are synthesized from poly(A)+ RNA using techniques well-known to those in the art. (see, for example, Ausubel (1990) at pages 5.5.2-5.6.8). Moreover, commercially available kits can be used to synthesize double-stranded cDNA molecules. For example, such kits are available from Life Technologies (Gaithersburg, Md.), Clontech Laboratories, Inc. (Palo Alto, Calif.), Promega Corporation (Madison, Wis.) and Stratagene Cloning Systems (La Jolla, Calif.).

Various cloning vectors are appropriate for the construction of a maize cDNA library. For example, a cDNA library can be prepared in a vector derived from bacteriophage, such as a λgt10 vector (see, for example, Huynh et al., “Constructing and Screening cDNA Libraries in λgt10 and λgt11,” in DNA Cloning: A Practical Approach Vol. I, Glover (ed.), page 49 (IRL Press, 1985)).

Alternatively, double-stranded cDNA molecules can be inserted into a plasmid vector, such as a pBluescript vector (Stratagene Cloning Systems; La Jolla, Calif.), a LambdaGEM-4 (Promega Corp.) or other commercially available vectors. Suitable cloning vectors also can be obtained from the American Type Culture Collection (Rockville, Md.).

In order to amplify the cloned cDNA molecules, the cDNA library is inserted into a prokaryotic host, using standard techniques. For example, a cDNA library can be introduced into competent E. coli DH5 cells, which can be obtained from Life Technologies, Inc. (Gaithersburg, Md.).

A plant genomic DNA library can be prepared by means well-known in the art (see, for example, Slightom et al. “Construction of λ Clone Banks,” in Methods in Plant Molecular Biology and Biotechnology, Glick et al. (eds.), page 121 (CRC Press 1993)). Genomic DNA can be isolated by lysing plant tissue with the detergent Sarkosyl, digesting the lysate with proteinase K, clearing insoluble debris from the lysate by centrifugation, precipitating nucleic acid from the lysate using isopropanol, and purifying resuspended DNA on a cesium chloride density gradient (see, for example, Ausubel (1990) at pages 2.3.1-2.3.3). Such methods are standard, and can be performed using commercially available kits, such as those sold by Gentra Systems (Minneapolis, Minn.).

DNA fragments that are suitable for the production of a genomic library can be obtained by the random shearing of genomic DNA or by the partial digestion of genomic DNA with restriction endonucleases (see, for example, Ausubel (1990) at pages 5.3.2-5.4.4, and Slightom et al., supra). Genomic DNA fragments can be inserted into a vector, such as a bacteriophage or cosmid vector, in accordance with conventional techniques, such as the use of restriction enzyme digestion to provide appropriate termini, the use of alkaline phosphatase treatment to avoid undesirable joining of DNA molecules, and ligation with appropriate ligases. Techniques for such manipulation are disclosed by Slightom et al., supra, and are well-known in the art (also see Ausubel (1990) at pages 3.0.5-3.17.5).

Alternatively, a plant genomic library can be obtained from a commercial source such as Clontech Laboratories, Inc, (Palo Alto, Calif.) or Stratagene Cloning Systems (La Jolla, Calif.).

A library containing cDNA or genomic clones can be screened with one or more polynucleotide probes based upon SEQ ID NO: 1 (see, for example, Ausubel (1990) at pages 6.0.3-6.6.1; Slightom et al., supra; Raleigh et al., Genetics 122:279, 1989).

As an alternative, a GFAT nucleic acid can be obtained by synthesizing DNA molecules using mutually priming long oligonucleotides (see, for example, Ausubel (1990) at pages 8.2.8 to 8.2.13 (1990); Wosnick et al., Gene 60:115, 1987; and Ausubel et al. (eds.), Short Protocols in Molecular Biology, 3rd Edition, pages 8-8 to 8-9 (John Wiley & Sons, Inc. 1995) [“Ausubel (1995)”]). Established techniques using the polymerase chain reaction provide the ability to synthesize DNA molecules at least two kilobases in length (Adang et al., Plant Molec. Biol. 21:1131, 1993); Bambot et al., PCR Methods and Applications 2:266, 1993); Dillon et al., “Use of the Polymerase Chain Reaction for the Rapid Construction of Synthetic Genes,” in Methods in Molecular Biology, Vol. 15: PCR Protocols: Current Methods and Applications, White (ed.), pages 263-268, (Humana Press, Inc. 1993); Holowachuk et al., PCR Methods Appl. 4:299, 1995).

3. Preparation of Variant GFAT Nucleic Acids

Additional nucleic acid molecules encoding GFAT nucleic acids can also be obtained by screening various cDNA or genomic libraries with polynucleotide probes having nucleotide sequences based upon SEQ ID NO: 1. Suitable libraries can be prepared by obtaining nucleic acids from tissue of any plant and constructing a library according to standard methods (see, for example, Ausubel (1995) at pages 2-1 to 2-13 and 5-1 to 5-6). Monocotyledonous plant species are preferred sources of nucleic acids. For example, nucleic acids can be obtained from tissues of wheat, barley, maize, rice, sorghum, or oats to construct libraries suitable for obtaining additional GFAT-encoding sequences.

Nucleic acid molecules that encode GFAT can also be obtained using the polymerase chain reaction (PCR) with oligonucleotide primers having nucleotide sequences that are based upon the nucleotide sequences of the maize GFAT nucleic acid, as described herein. General methods for screening libraries with PCR are provided by, for example, Yu et al., “Use of the Polymerase Chain Reaction to Screen Phage Libraries,” in Methods in Molecular Biology, Vol. 15: PCR Protocols: Current Methods and Applications, White (ed.), pages 211-215 (Humana Press, Inc. 1993). Moreover, techniques for using PCR to isolate related genes are described by, for example, Preston, “Use of Degenerate Oligonucleotide Primers and the Polymerase Chain Reaction to Clone Gene Family Members,” in Methods in Molecular Biology, Vol. 15: PCR Protocols: Current Methods and Applications, White (ed.), pages 317-337 (Humana Press, Inc. 1993). One illustration of this general approach is described by Sayeski et al, Gene 140:289 (1994), who prepared a murine cDNA library using primers based on the sequence of a human GFAT gene.

Anti-GFAT antibodies, produced as described below, can also be used to isolate DNA sequences that encode enzymes from cDNA libraries constructed from mRNA from various species. For example, the antibodies can be used to screen λgt11 expression libraries, or the antibodies can be used for immunoscreening following hybrid selection and translation (see, for example, Ausubel (1995) at pages 6-12 to 6-16; and Margolis et al., “Screening λ expression libraries with antibody and protein probes,” in DNA Cloning 2: Expression Systems, 2nd Edition, Glover et al. (eds.), pages 1-14 (Oxford University Press 1995)).

GFAT nucleic acid variants can also be constructed synthetically. For example, a nucleic acid molecule can be devised that encodes a polypeptide having a conservative amino acid change, compared with the amino acid sequence of SEQ ID NO: 2. That is, variants can be obtained that contain one or more amino acid substitutions of SEQ ID NO: 2, in which an alkyl amino acid is substituted for an alkyl amino acid in the maize GFAT amino acid sequence, an aromatic amino acid is substituted for an aromatic amino acid in the maize GFAT amino acid sequence, a sulfur-containing amino acid is substituted for a sulfur-containing amino acid in the maize GFAT amino acid sequence, a hydroxy-containing amino acid is substituted for a hydroxy-containing amino acid in the maize GFAT amino acid sequence, an acidic amino acid is substituted for an acidic amino acid in the maize GFAT amino acid sequence, a basic amino acid is substituted for a basic amino acid in the maize GFAT amino acid sequence, or a dibasic monocarboxylic amino acid is substituted for a dibasic monocarboxylic amino acid in the maize GFAT amino acid sequence.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine, threonine, methionine, cystine, and alanine, (4) aspartate and glutamate, (5) glutamine and asparagine, and (6) lysine, arginine and histidine.

Conservative amino acid changes in the maize GFAT nucleic acid can be introduced by substituting nucleotides for the nucleotides recited in SEQ ID NO: 1. Such “conservative amino acid” variants can be obtained, for example, by oligonucleotide-directed mutagenesis, linker-scanning mutagenesis, mutagenesis using the polymerase chain reaction, and the like (see Ausubel (1990) at pages 8.0.3-8.5.9; Ausubel (1995) at pages 8-10 to 8-22; and McPherson (ed.), Directed Mutagenesis: A Practical Approach (IRL Press 1991)). The ability of such variants to convert fructose-6-phosphate to glucosamine-6-phosphate can be determined using a standard enzyme activity assay, such as one of the assays described herein.

In addition, routine deletion analyses of nucleic acid molecules can be performed to obtain “functional fragments” of a nucleic acid molecule that encodes GFAT. As an illustration, DNA molecules having the nucleotide sequence of SEQ ID NO: 1 can be digested with Bal31 nuclease to obtain a series of nested deletions. The fragments are then inserted into expression vectors in proper reading frame, and the expressed polypeptides are isolated and tested for GFAT enzyme activity. One alternative to exonuclease digestion is to use oligonucleotide-directed mutagenesis to introduce deletions or stop codons to specify production of a desired fragment. Alternatively, particular fragments of a maize GFAT nucleic acid can be synthesized using the polymerase chain reaction. Standard techniques for functional analysis of proteins are described by, for example, Treuter et al., Molec. Gen. Genet 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation, Vol. 1, Boynton et al., (eds.) pages 169-199 (Academic Press 1985); Coumailleau et al., J. Biol. Chem. 270:29270, 1995; Fukunaga et al., J. Biol. Chem. 270:25291, 1995; Yamaguchi et al., Biochem. Pharmacol. 50:1295, 1995; and Meisel et al., Plant Molec. Biol. 30:1, 1996. The present invention also contemplates functional fragments of a GFAT nucleic acid that have conservative amino acid changes.

Furthermore, deletions and/or insertions of the GFAT nucleic acid can be constructed by any of a variety of known methods. For example, the nucleic acid can be digested with restriction enzymes and religated such that the resultant sequence lacks a sequence of the native nucleic acid, or religated with an additional DNA fragment such that the resultant sequence contains an insertion or large substitution. Other standard methods for generating variant sequences may be used as described, for example, by Sambrook and Ausubel (1995). Verification of variant sequences is typically accomplished by restriction enzyme mapping, sequence analysis, or probe hybridization.

4. Expression of Cloned GFAT Nucleic Acids

To express the polypeptide encoded by a GFAT nucleic acid, a nucleotide sequence encoding the enzyme must be operably linked to nucleotide sequences that control transcriptional expression in an expression vector and then, introduced into either a prokaryotic or eukaryotic host cell. In addition to nucleotide sequences that control transcription, such as promoters and regulatory elements, expression vectors can include translational regulatory sequences, and a marker gene that is suitable for selection of cells that carry the expression vector.

Depending on the desired use of an expressed GFAT polypeptide, it may be advantageous to produce GFAT polypeptide as a fusion protein. For example, a fusion protein can be expressed that comprises both GFAT sequences and a portion that binds with an affinity matrix. In this way, large quantities of GFAT polypeptides can be obtained by cleaving the polypeptides from fusion protein bound to an affinity chromatography column. Alternatively, it may be desirable to express a fusion protein comprising a GFAT sequence and a transit peptide for targeting the enzyme to a particular organelle. Such transit peptides are discussed below. Accordingly, the present invention contemplates fusion proteins comprising GFAT polypeptides.

Suitable promoters for expression of GFAT polypeptides in a prokaryotic host can be repressible, constitutive, or inducible. Suitable promoters are well-known to those of skill in the art and include promoters capable of recognizing the T4, T3, Sp6 and T7 polymerases, the P_{R} and P_{L} promoters of bacteriophage lambda, the trp, recA, heat shock, lacUV5, tac, lpp-lacSpr, phoA, and lacZ promoters of E. coli, promoters of B. subtilis, the promoters of the bacteriophages of Bacillus, Streptomyces promoters, the int promoter of bacteriophage lambda, the bla promoter of pBR322, and the CAT promoter of the chloramphenicol acetyl transferase gene. Prokaryotic promoters are reviewed by Glick, J. Ind. Microbiol. 1:277 (1987); Watson et al., Molecular Biology of the Gene, 4th Ed. (Benjamin Cummins 1987); Ausubel et al. (1990, 1995), and Sambrook et al., supra.

Preferred prokaryotic hosts include E. coli and Bacillus subtilus. Suitable strains of E. coli include BL21 (DE3), BL21 (DE3)pLysS, BL21 (DE3)pLysE, DH1, DH4I, DH5, DH5I, DH5IF′, DH5IMCR, DH10B, DH10B/p3, DH11S, C600, HB101, JM101, JM105, JM109, JM110, K38, RR1, Y1088, Y1089, CSH18, ER1451, and ER1647 (see, for example, Brown (Ed.), Molecular Biology Labfax, Academic Press (1991)). Suitable strains of Bacillus subtilus include BR151, YB886, MI119, MI120, and B170 (see, for example, Hardy, “Bacillus Cloning Methods,” in DNA Cloning: A Practical Approach, Glover (Ed.), (IRL Press 1985)).

Methods for expressing proteins in prokaryotic hosts are well-known to those of skill in the art (see, for example, Williams et al., “Expression of foreign proteins in E. coli using plasmid vectors and purification of specific polyclonal antibodies,” in DNA Cloning 2: Expression Systems, 2nd Edition, Glover et al., (eds.), page 15 (Oxford University Press 1995); Ward et al., “Genetic Manipulation and Expression of Antibodies,” in Monoclonal Antibodies: Principles and Applications, page 137 (Wiley-Liss, Inc. 1995); and Georgiou, “Expression of Proteins in Bacteria,” in Protein Engineering: Principles and Practice, Cleland et al. (eds.), page 101 (John Wiley & Sons, Inc. 1996)).

Expression vectors also can be introduced into eukaryotic hosts, such as mammalian cells, yeast cells, insect cells, and plant cells. Expression vectors that are suitable for production of GFAT protein in eukaryotic cells typically contain (1) prokaryotic DNA elements coding for a bacterial replication origin and an antibiotic resistance marker to provide for the growth and selection of the expression vector in a bacterial host; (2) eukaryotic DNA elements that control initiation of transcription, such as a promoter; and (3) DNA elements that control the processing of transcripts, such as a transcription termination/polyadenylation sequence.

Examples of mammalian host cells include human embryonic kidney cells (293-HEK; ATCC CRL 1573), baby hamster kidney cells (BHK-21; ATCC CRL 8544), canine kidney cells (MDCK; ATCC CCL 34), Chinese hamster ovary cells (CHO-K1; ATCC CCL61), rat pituitary cells (GH₁; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Preferably the mammalian host cells are other than human.

For a mammalian host, the transcriptional and translational regulatory signals may be derived from viral sources, such as adenovirus, bovine papilloma virus, simian virus, or the like, in which the regulatory signals are associated with a particular gene which has a high level of expression. Suitable transcriptional and translational regulatory sequences also can be obtained from mammalian genes, such as actin, collagen, myosin, and metallothionein genes.

Transcriptional regulatory sequences include a promoter region sufficient to direct the initiation of RNA synthesis. Suitable eukaryotic promoters include the promoter of the mouse metallothionein I gene [Hamer et al., J. Molec. Appl. Genet. 1:273, 1982)], the TK promoter of Herpes virus [McKnight, Cell 31:355, 1982)], the SV40 early promoter [Benoist et al, Nature 290:304, 1981)], the Rous sarcoma virus promoter [Gorman et al., Proc. Nat'l Acad. Sci. USA 79:6777, 1982), the cytomegalovirus promoter [Foecking et al., Gene 45:101, 1980)], and the mouse mammary tumor virus promoter. See, generally, Etcheverry, “Expression of Engineered Proteins in Mammalian Cell Culture,” in Protein Engineering: Principles and Practice, Cleland et al. (eds.), pages 163-181 (John Wiley & Sons, Inc. 1996).

Alternatively, a prokaryotic promoter, such as the bacteriophage T3 RNA polymerase promoter, can be used to control fusion gene expression if the prokaryotic promoter is regulated by a eukaryotic promoter (see, for example, Zhou et al., Mol. Cell. Biol. 10:4529, 1990; Kaufman et al., Nucl. Acids Res. 19:4485, 1991).

The baculovirus system provides an efficient means to introduce cloned GFAT nucleic acids into insect cells. Suitable expression vectors are based upon the Autographa californica multiple nuclear polyhedrosis virus (AcMNPV), and contain well-known promoters such as Drosophila heat shock protein (hsp) 70 promoter, Autographa californica nuclear polyhedrosis virus immediate-early gene promoter (ie-1) and the delayed early 39K promoter, baculovirus p10 promoter, and the Drosophila metallothionein promoter. Suitable insect host cells include cell lines derived from IPLB-Sf-21, a Spodoptera frugiperda pupal ovarian cell line, such as Sf9 (ATCC CRL 1711), Sf21AE, and Sf21 (Invitrogen Corporation; San Diego, Calif.), as well as Drosophila Schneider-2 cells. Established techniques for producing recombinant proteins in baculovirus systems are provided by Bailey et al., “Manipulation of Baculovirus Vectors,” in Methods in Molecular Biology, Volume 7: Gene Transfer and Expression Protocols, Murray (ed.), pages 147-168 (The Humana Press, Inc. 1991), by Patel et al., “The baculovirus expression system,” in DNA Cloning 2: Expression Systems, 2nd Edition, Glover et al. (eds.), pages 205-244 (Oxford University Press 1995), by Ausubel (1995) at pages 16-37 to 16-57, by Richardson (ed.), Baculovirus Expression Protocols (The Humana Press, Inc. 1995), and by Lucknow, “Insect Cell Expression Technology,” in Protein Engineering: Principles and Practice, Cleland et al. (eds.), pages 183-218 (John Wiley & Sons, Inc. 1996).

Promoters for expression in yeast include promoters from GALL (galactose), PGK (phosphoglycerate kinase), ADH (alcohol dehydrogenase), AOX1 (alcohol oxidase), HIS4 (histidinol dehydrogenase), and the like. Many yeast cloning vectors have been designed and are readily available. These vectors include YIp-based vectors, such as YIp5, YRp vectors, such as YRp17, YEp vectors such as YEp13 and YCp vectors, such as YCp19. One skilled in the art will appreciate that there are a wide variety of suitable vectors for expression in yeast cells.

With respect to plants, examples of seed-preferred promoters include promoters of seed storage proteins which express these proteins in seeds in a highly regulated manner (Thompson, et al.; BioEssays. 10:108; (1989), incorporated herein in its entirety by reference), such as, for dicotyledonous plants, a bean β-phaseolin promoter, a napin promoter, a β-conglycinin promoter, and a soybean lectin promoter. For monocotyledonous plants, promoters useful in the practice of the invention include, but are not limited to, a maize 15 kD zein promoter, a 22 kD zein promoter, a γ-zein promoter, a waxy promoter, a shrunken 1 promoter, a globulin 1 promoter, and the shrunken 2 promoter. However, other promoters useful in the practice of the invention are known to those of skill in the art.

Constitutive, tissue-preferred or inducible promoters can be employed. Examples of constitutive promoters include the cauliflower mosaic virus (CaMV) 35S transcription initiation region, the 1′- or 2′-promoter derived from T-DNA of Agrobacterium tumefaciens, the ubiquitin 1 promoter, the Smas promoter, the cinnamyl alcohol dehydrogenase promoter (U.S. Pat. No. 5,683,439), the Nos promoter, the pEmu promoter, the rubisco promoter, the GRP1-8 promoter and other transcription initiation regions from various plant genes known to those of skill.

Examples of inducible promoters are the Adh1 promoter which is inducible by hypoxia or cold stress, the Hsp70 promoter which is inducible by heat stress, and the PPDK promoter which is inducible by light. Also useful are promoters which are chemically inducible.

Examples of promoters under developmental control include promoters that initiate transcription preferentially in certain tissues, such as leaves, roots, fruit, seeds, or flowers. An exemplary promoter is the anther specific promoter 5126 (U.S. Pat. Nos. 5,689,049 and 5,689,051). Examples of seed-preferred promoters include, but are not limited to, 27 kD gamma zein promoter and waxy promote, Boronat, A., Martinez, M. C., Reina, M., Puigdomenech, P. and Palau, J.; Isolation and sequencing of a 28 kD glutelin-2 gene from maize: Common elements in the 5′ flanking regions among zein and glutelin genes; Plant Sci. 47, 95-102 (1986) and Reina, M., Ponte, I., Guillen, P., Boronat, A. and Palau, J., Sequence analysis of a genomic clone encoding a Zc2 protein from Zea mays W64 A, Nucleic Acids Res. 18 (21), 6426 (1990). See the following site relating to the waxy promoter: Kloesgen, R. B., Gierl, A., Schwarz-Sommer, Z S. and Saedler, H., Molecular analysis of the waxy locus of Zea mays, Mol. Gen. Genet 203:237-244 (1986). Promoters that express in the embryo, pericarp, and endosperm are disclosed in U.S. applications Ser. No. 60/097,233 filed Aug. 20, 1998 and No. 60/098,230 filed Aug. 28, 1998. The disclosures each of these are incorporated herein by reference in their entirety.

Other examples of suitable promoters are the promoter for the small subunit of ribulose-1,5-bis-phosphate carboxylase, promoters from tumor-inducing plasmids of Agrobacterium tumefaciens, such as the nopaline synthase and octopine synthase promoters, and viral promoters such as the cauliflower mosaic virus (CaMV) 19S and 35S promoters or the figwort mosaic virus 35S promoter.

Either heterologous or non-heterologous (i.e., endogenous) promoters can be employed to direct expression of the nucleic acids of the present invention. These promoters can also be used, for example, in expression cassettes to drive expression of antisense nucleic acids to reduce, increase, or alter concentration and/or composition of the proteins of the present invention in a desired tissue.

A GFAT expression vector can also include a nucleotide sequence encoding a secretion signal. In this way, recombinant GFAT protein can be recovered from the periplasmic space of host cells or from fermentation medium. Secretion signals suitable for use are widely available and are well-known in the art (see, for example, von Heijne, J. Mol. Biol. 184:99, 1985). Prokaryotic and eukaryotic secretion signals that are functional in E. coli (or other host cells) may be employed. Suitable secretion signals include, but are not limited to, those encoded by the following E. coli genes: pelB, phoA, ompA, ompT, ompF, ompC, beta-lactamase, and alkaline phosphatase (see, for example, Lei et al., J. Bacteriol. 169:4379, 1987). As a further example, the signal sequence from the cek2 gene is useful for secretion in recombinant insect cells Those of skill in the art are aware of secretion signals that are functional in prokaryotic, yeast, insect or mammalian cells to secrete proteins from those cells.

An expression vector can be introduced into bacterial, mammalian, insect, and yeast host cells using a variety of techniques including calcium chloride transformation, liposome-mediated transfection, electroporation, and the like (see, for example, Ausubel (1995) at pages 1-1 to 1-24). Preferably, transfected cells are selected and propagated wherein the expression vector is stably integrated in the host cell genome to produce stable transformants. Techniques for introducing vectors into eukaryotic cells and techniques for selecting stable transformants using a dominant selectable marker are described, for example, by Ausubel (1990, 1995) and by Murray, supra.

Expression vectors can also be introduced into plant protoplasts, intact plant tissues, or isolated plant cells. General methods of culturing plant tissues are provided, for example, by Miki et al., “Procedures for Introducing Foreign DNA into Plants,” in Methods in Plant Molecular Biology and Biotechnology, Glick et al. (eds.), pages 67-88 (CRC Press, 1993). Methods of introducing expression vectors into plant tissue include the direct infection or co-cultivation of plant tissue with Agrobacterium tumefaciens (see, for example, Horsch et al., Science 227:1229, 1985). Descriptions of Agrobacterium vector systems and methods for Agrobacterium-mediated gene transfer are provided by Gruber et al., “Vectors for Plant Transformation,” in Methods in Plant Molecular Biology and Biotechnology, Glick et al. (eds.), pages 89-119 (CRC Press 1993), by Miki et al., supra, and by Moloney et al., Plant Cell Reports 8:238, 1989.

Alternatively, expression vectors are introduced into plant tissues using a direct gene transfer method such as microprojectile-mediated delivery, DNA injection, electroporation, and the like (see, for example, Gruber et al., supra; Miki et al., supra; Klein et al., Biotechnology 10:268, 1992). For example, expression vectors can be introduced into various plant tissues using microprojectile-mediated delivery with a biolistic device (see, generally, Yang and Christou (eds.), Particle Bombardment Technology for Gene Transfer (Oxford University Press 1994)).

5. Isolation of Cloned GFAT Enzymes, Measurement of Enzyme Activity, and Production of Anti-GFAT Antibodies

(a) Isolation of Cloned GFAT Protein

General methods for recovering protein produced by a recombinant host are well-known to those of skill in the art. For example, standard techniques for isolation of protein from a bacterial system are provided by Grisshammer et al., “Purification of over-produced proteins from E. coli cells,” in DNA Cloning 2: Expression Systems, 2nd Edition, Glover et al. (eds.), pages 59-92 (Oxford University Press 1995); Georgiou, “Expression of Proteins in Bacteria,” in Protein Engineering: Principles and Practice, Cleland et al. (eds.), pages 101-127 (Wiley-Liss, Inc. 1996). Established techniques for isolating recombinant proteins from a baculovirus system are described by Richardson (ed.), Baculovirus Expression Protocols (The Humana Press, Inc. 1995). More generally, GFAT protein can be isolated by standard techniques, such as affinity, chromatography, size exclusion chromatography, ionic exchange chromatography, HPLC and the like. An isolated protein should show a single band by Coomassie blue stain of a gel following SDS-PAGE. Additional variations in enzyme isolation and purification can be devised by those of skill in the art. For example, anti-GFAT antibodies, obtained as described below, can be used to isolate large quantities of enzyme by immunoaffinity purification.

As an illustration, the GFAT cDNA described herein can be cloned into pET-22 cloning vector obtained from Novagen Incorporated (Madison, Wis.). Protein is produced by the vector manufacturer's recommended protocols, and the resultant GFAT protein is recovered as insoluble protein bodies. The protein bodies are washed by re-suspension in 15 mM Tris•Cl (pH 7.4) and 0.1% Triton X-100. After vortexing, the protein bodies are recovered by centrifugation at 10,000×G for five minutes. The wash and collection cycle are repeated three times.

Recovered protein can be further purified by polyacrylamide gel electrophoresis, performed according to standard protocols. The protein band corresponding to GFAT is identified by brief amido black staining, and the band is cut from the gel. The protein may be electroeluted, according to established methods. Alternatively, the protein may be used directly as antigen for producing antibodies.

Isolated GFAT protein, obtained from recombinant hosts, can be used to produce polysaccharide precursors in vitro. Moreover, GFAT enzyme from cloned GFAT nucleic acids is useful for the stereospecific production of glucosamine-6-phosphate as a fine chemical. For example, a preparation of isolated polypeptide having GFAT enzyme activity can be used to synthesize stereospecifically-labeled tritiated glucosamine-6-phosphate.

(b) Assays for Variant and Mutant GFAT Enzymes

GFAT enzyme activity can be determined using standard in vitro methods. Typically, a sample containing GFAT enzyme is incubated with substrate and the product, glucosamine-6-phosphate is measured with a standard assay, such as a colormetric assay. For example, Vessal and Hassid, Plant Physiol. 49:977 (1972), describe an assay for mung bean GFAT in which a sample containing GFAT enzyme is mixed with D-fructose-6-phospate and L-glutamine, incubated at 30° C. for 1.5 hours, and boiled for two minutes to stop the reaction. Following centrifugation, an aliquot of the supernatant was analyzed for D-glucosamine-6-phosphate using a modification of the colormetric assay of Ghosh et al., J. Biol. Chem. 235:1265, 1960.

As an alternative, GFAT enzyme activity can be measured using a radioenzymatic assay in which the enzyme converts radiolabeled fructose-6-phosphate to radiolabeled glucosamine-6-phosphate.

In a preferred method, a protein sample (typically, about 3 μg total protein/reaction) is incubated in a solution containing 60 mM KH₂PO₄ (pH 7.0), 1 mM EDTA, 1 mM DTT, 15 mM glutamine, and 15 mM fructose-6-phosphate. The reaction is incubated at 30° C. for 1 hour, and stopped by boiling five minutes. The mixture is then centrifuged at 14,000×g for 2 minutes, the supernatant is transferred to a Microcon-30, and then centrifuged at 14,000×g for 10 minutes at 4° C. to separate proteins from sugars in the flow-through. The filtrate is analyzed using high pressure liquid chromatography (HPLC) and the retentate is discarded. HPLC is used to separate fructose-6-phosphate and glucosamine-6-phosphate.

(c) Preparation of Anti-GFAT Antibodies and Fragments Thereof

Antibodies to GFAT can be obtained, for example, using the product of an expression vector as an antigen. Polyclonal antibodies to recombinant enzyme can be prepared using methods well-known to those of skill in the art (see, for example, Green et al., “Production of Polyclonal Antisera,” in Immunochemical Protocols (Manson, ed.), pages 1-5 (Humana Press 1992); Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995)).

Alternatively, an anti-GFAT antibody can be derived from a rodent monoclonal antibody. Rodent monoclonal antibodies to specific antigens may be obtained by methods known to those skilled in the art (see, for example, Kohler et al., Nature 256:495, 1975; and Coligan et al. (eds.), Current Protocols in Immunology, Vol. 1, pages 2.5.1-2.6.7 (John Wiley & Sons 1991) [“Coligan”]; Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising an antigen, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-GFAT antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. Nos. 4,036,945 and 4,331,647 and references contained therein. Also, see Nisonoff et al., Arch Biochem. Biophys. 89:230, 1960; Porter, Biochem. J. 73:119, 1959; Edelman et al., in Methods in Enzymology Vol. 1, page 422 (Academic Press 1967); Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437, 1992).

Preferably, the Fv fragments comprise V_{H} and V_{L} chains which are connected by a peptide linker. These single-chain antigen binding proteins (sFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing sFvs are described, for example, by Whitlow et al., Methods: A Companion to Methods in Enzymology 2:97 (1991). Also see Bird et al., Science 242:423 (1988), Ladner et al., U.S. Pat. No. 4,946,778, Pack et al., Bio/Technology 11:1271 (1993), and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (See, for example, Larrick et al., Methods: A Companion to Methods in Enzymology 2:106 (1991); Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); and Ward et al., “Genetic Manipulation and Expression of Antibodies,” in Monoclonal Antibodies: Principles and Applications, Birch et al., (eds.), page 137 (Wiley-Liss, Inc. 1995)).

6. Modification of Polysaccharide Biosynthesis in Transgenic Plants That Express an Exogenous GFAT Nucleic Acid

(a) Production of Transgenic Plants That Express an Exogenous GFAT Nucleic Acid

In order to alter plant polysaccharide biosynthesis, an expression vector is constructed in which a nucleotide sequence encoding a GFAT nucleic acid is operably linked to nucleotide sequences that regulate gene transcription. The general requirements of an expression vector are described above in the context of a transient expression system. Here, however, the objective is to introduce the expression vector into plant embryonic tissue in such a manner that a GFAT enzyme will be expressed in tissues of the adult plant. One method of obtaining mitotic stability is provided by the integration of expression vector sequences into the host chromosome. Such mitotic stability can be provided, for example, by microprojectile bombardment or by Agrobacterium-mediated transformation.

Transcription of a GFAT nucleic acid in a transgenic plant can be controlled by a viral promoter, such as a Cauliflower Mosaic Virus (CaMV) promoter and a Figwort Mosaic Virus promoter. Additional useful promoters include ubiquitin promoters, mannopine synthase promoters, DNAJ, GST-responsive promoters, and heat shock gene promoters (e.g., hsp70). Regulatory elements that provide tissue-specific gene expression are also useful. Such regulatory elements include, for example, seed-specific regulatory elements, such as maize zein or waxy regulatory elements, napin regulatory elements (U.S. Pat. No. 5,420,034), cruciferin regulatory elements from canola, helianthianin regulatory elements from sunflower, the α′-conglycinin subunit regulatory elements from soybean, Bce4 regulatory elements (U.S. Pat. No. 5,530,194), or regulatory elements from genes of other seed storage proteins (see, for example, Gruber et al., supra). Additional suitable regulatory elements are well-known to those of skill in the art.

Depending upon the application, it may be desirable to select promoters that are not constitutive but specific for expression in one or more tissues of the plant. Such examples include the light-inducible promoters of the small subunit of ribulose 1,5-bisphosphate carboxylase, if the expression is desired in photosynthetic tissues, or promoters of seed-specific genes, as noted above. In addition, specific timing of expression may be desirable. In this regard, chemically-inducible promoters are known in the art which allow the controlled expression of a gene of interest at a specific stage of development (see, for example, Hershey et al., international publication No. WO 90/11361).

Particularly preferred regulatory elements and promoters are those that allow seed-specific expression. Examples of seed-specific regulatory elements and promoters include but are not limited to nucleotide sequences that control expression of seed storage proteins, which can represent up to 90% of total seed protein in many plants. The seed storage proteins are strictly regulated, being expressed almost exclusively in seeds in a highly tissue-specific and stage-specific manner (see, for example, Higgins et al., Ann. Rev. Plant Physiol. 35:191, 1984; Goldberg et al., Cell 56:149, 1989). Moreover, different seed storage proteins may be expressed at different stages of seed development. Expression of seed-specific genes has been studied in great detail (see, for example, Goldberg et al., Cell 56:149, 1989; Higgins et al., Ann. Rev. Plant Physiol. 35:191, 1984).

As discussed above, this invention provides the expression in plants of a GFAT nucleic acid under control of a promoter, and optionally, a regulatory element, such as an organelle-specific, cell-specific, or tissue-specific regulatory element. The choice of the promoter and a regulatory element will depend in part upon the desired result.

In certain embodiments, the vector can also contain a reporter gene and a GFAT nucleic acid. The inclusion of a reporter gene allows determination of transformation and expression. The GUS (β-glucoronidase) gene is preferred (see, for example, U.S. Pat. No. 5,268,463). Other reporter genes, such as β-galactosidase, luciferase, green fluorescent protein, and the like, are also suitable in the context of this invention. Methods and substrates for assaying expression of each of these genes are well known in the art. The reporter gene should be under control of a promoter that is functional in plants. Such promoters include CaMV 35S promoter, mannopine synthase promoter, ubiquitin promoter and DNA J promoter.

Particular uses for GFAT expression may require additional regulatory elements, as discussed below. For example, an expression vector can include a nucleotide sequence that encodes a transit peptide or a signal sequence joined with GFAT-encoding sequences. Transit peptides enable the translocation of a nuclear encoded polypeptide into the chloroplast or the mitochondria, while signal sequences direct an associated protein into the lumen of the endoplasmic reticulum. During the maturation process, the transit peptide or signal sequence is removed from the protein. Plant transit sequences and signal sequences are well-known in the art (see, for example, Keegstra and Olsen, Annu. Rev. Plant Mol. Biol. 40:471, 1989).

As an illustration, the transit peptide of the small subunit of the enzyme 1,5-ribulose bisphosphate carboxylase enables transport into chloroplasts. This peptide and other chloroplast transit peptides can also be used in the present invention (see, for example, Krebbers et al., Plant Mol. Biol. 11:745, 1988; European patent application No. 85402596.2; Watson, Nucl. Acids Res. 12:5145, 1984; Yon Heijne et al., Plant Mol. Biol. Rep. 9:104, 1991). Suitable mitochondrial targeting peptides include the mitochondrial transit peptides described by Schatz, Eur. J. Biochem. 165:1 (1987), and listed by Watson, supra. Suitable signal sequences that can translocate a protein of interest to the lumen of the endoplasmatic reticulum of a plant cell include, for example, the signal sequences described by Von Heijne, Biochem. Biophys. Acta 947:307 (1988), and by Watson, supra.

In general, transit peptide sequences obtained from any polypeptide that is transported into plastids can be used to direct the GFAT nucleic acid product to the desired subcellular compartment. Preferred transit peptides include sequences associated with the following genes: brittle-1, small subunit of ribulose 1,5-bisphospate carboxylase, and light harvesting chlorophyll protein. Suitable amino acid sequences of transit peptides are well-known to those of skill in the art (see, for example, Sullivan et al., Plant Cell 3:1337, 1991; Gosh et al., Photochem. Photobiol. 57:352, 1993; Gotor et al., Plant J. 3:509, 1993; Sullivan, Planta 196:477, 1995; Pear et al., Proc. Nat'l Acad. Sci. USA 93:12637, 1996).

For example, the transit peptide sequence of the brittle-1 gene, which directs the associated polypeptide into amyloplasts, is described by Sullivan et al., Plant Cell 3:1337 (1991), and by Li et al., Journal of Biological Chemistry 267:18999 (1992). A suitable brittle-1 transit peptide is encoded by the following nucleotide sequence which includes additional amino acids (encoded by nucleotides 226-237) to preserve protease cleavage junction integrity:

| | | |
|---|---|---|
| 1 | ATGGCGGCGA CAATGGCAGT GACGACGATG GTGACCAGGA GCAAGGAGAG | [SEQ ID NO: 3] |
| 51 | CTGGTCGTCA TTGCAGGTCC CGGCGGTGGC ATTCCCTTGG AAGCCACGAG | |
| 101 | GTGGCAAGAC CGGCGGCCTC GAGTTCCCTC GCCGGGCGAT GTTCGCCAGC | |
| 151 | GTCGGCCTCA ACGTGTGCCC GGGCGTCCCG GCGGGGCGCG ACCCGCGGGA | |
| 201 | GCCCGATCCC AAGGTCGTCC GGGCGGCCGA CCTCATG. | |

In order to select transformed cells, an expression vector can contain a selectable marker gene, such as a herbicide resistance gene or an antibiotic resistance gene. For example, such genes may confer resistance to phosphinothricin, glyphosate, sulfonylureas, atrazine, imidazolinone or aminoglycoside antibiotics such as neomycin, kanamycin and G418 (genticin). Preferred selectable marker genes are the neomycin phosphotransferase gene (nptII gene), and the bar gene or pat gene which encodes phosphinothricin acetyltransferase. The nucleotide sequences of bar genes can be found in Leemans et al., European patent application No. 0-242-246 (1987), and in White et al., Nucleic Acids Res. 18: 1062, 1990. Wohlleben et al., Gene 70: 25 (1988), disclose the nucleotide sequence of the pat gene. Bar or pat gene expression confers resistance to herbicides such as glufosinate (sold as Basta® and Ignite®, among others) and bialaphos (sold as Herbi-ace® and Liberty®).

The expression vector can contain nucleotide sequences encoding a GFAT protein under the control of a promoter, and optionally, a regulatory element, as well as the selectable marker gene under control of a constitutive promoter. Alternatively, the selectable marker gene can be delivered to host cells in a separate selection expression vector by co-transformation with both vectors.

Any plant that would benefit from either expression of a GFAT nucleic acid or inhibition of GFAT activity is suitable for transformation within the context of this invention. Such plants include maize, sorghum, wheat, rice, barley, oats, sunflower, soybean, Brassica, cassava, sweet potato, potato and the like.

A GFAT nucleic acid can be introduced into a plant using an established method. As noted above, Agrobacterium-mediated transformation can be used to produce such transgenic plants. This approach is illustrated in Example 1.

Alternatively, transgenic plants can be produced by microprojectile bombardment. For example, transgenic maize plants can be produced by bombardment of embryogenically responsive immature embryos with tungsten particles associated with plasmids as follows. About 15 milligrams of tungsten particles (General Electric), 0.5 to 1.8μ in diameter, preferably 1 to 1.8μ, and most preferably 1μ, are added to 2 ml of concentrated nitric acid. This suspension is sonicated at 0° C. for 20 minutes (Branson Sonifier Model 450, 40% output, constant duty cycle). Tungsten particles are pelleted by centrifugation at 10,000 rpm (Biofuge) for one minute, and then supernatant is removed. Two milliliters of sterile distilled water are added to the pellet, and brief sonication is used to resuspend the particles. The suspension is pelleted, one milliliter of absolute ethanol is added to the pellet, and brief sonication is used to resuspend the particles. Rinsing, pelleting, and resuspending of the particles is performed two more times with sterile distilled water, and finally the particles are resuspended in two milliliters of sterile distilled water. The particles are subdivided into 250-ml aliquots and stored frozen.

To coat particles with plasmid DNA that comprise a GFAT nucleic acid, the stock of tungsten particles is sonicated briefly in a water bath sonicator (Branson Sonifier Model 450, 20% output, constant duty cycle) and 50 μl are transferred to a microfuge tube. Equimolar amounts of plasmid DNA encoding a selectable marker gene and a GFAT nucleic acid are added to the particles for a final DNA amount of 0.1 to 10 μg in 10 μl total volume, and briefly sonicated. Preferably, 1 μg total DNA is used. For example, aliquots of an expression vector comprising the bar gene and an expression vector comprising a GFAT nucleic acid, both at 0.1 mg/ml in TE buffer, are added to the particle suspension. Fifty microliters of sterile aqueous 2.5 M CaCl₂ are added, and the mixture is briefly sonicated and vortexed. Twenty microliters of sterile aqueous 0.1 M spermidine are then added and the mixture is briefly sonicated and vortexed. The mixture is incubated at room temperature for 20 minutes with intermittent brief sonication. The particle suspension is centrifuged, and the supernatant is removed. Two hundred fifty microliters of absolute ethanol are added to the pellet, followed by brief sonication. The suspension is pelleted, the supernatant is removed, and 60 ml of absolute ethanol are added. The suspension is sonicated briefly before loading the particle-DNA agglomeration onto macrocarriers.

Immature embryos of maize variety High Type II are an example of a suitable target for particle bombardment-mediated transformation. This genotype is the F₁ of two purebred genetic lines, parents A and B, derived from the cross of two known maize inbreds, A188 and B73. Both parents are selected for high competence of somatic embryogenesis, according to Armstrong et al., Maize Genetics Coop. News 65:92 (1991).

Ears from F₁ plants are selfed or sibbed, and embryos are aseptically dissected from developing caryopses when the scutellum first becomes opaque. This stage occurs about 9-13 days post-pollination, and most generally about 10 days post-pollination, depending on growth conditions. The embryos are about 0.75 to 1.5 millimeters long. Ears are surface-sterilized with 20-50% Clorox for 30 minutes, followed by three rinses with sterile distilled water.

Immature embryos are cultured with the scutellum oriented upward, on embryogenic induction medium comprised of N6 basal salts, Eriksson vitamins, 0.5 mg/l thiamine HCl, 30 gm/l sucrose, 2.88 gm/l L-proline, 1 mg/l 2,4-dichlorophenoxyacetic acid, 2 gm/l Gelrite, and 8.5 mg/l silver nitrate (see, for example, Chu et al., Sci. Sin. 18:659, 1975; Eriksson, Physiol. Plant 18:976, 1965). The medium is sterilized by autoclaving at 121° C. for 15 minutes and dispensed into 100×25 mm Petri dishes. Silver nitrate is filter-sterilized and added to the medium after autoclaving. The tissues are cultured in complete darkness at 28° C. After about 3 to 7 days, most usually about 4 days, the scutellum of the embryo has swelled to about double its original size and the protuberances at the coleorhizal surface of the scutellum indicates the inception of embryogenic tissue. Up to 100% of the embryos may display this response, but most commonly, the embryogenic response frequency is about 80%.

When the embryogenic response is observed, the embryos are transferred to a medium comprised of induction medium modified to contain 120 gm/l sucrose. The embryos are oriented with the coleorhizal pole, the embryogenically responsive tissue, upwards from the culture medium. Ten embryos per Petri dish are located in the center of a Petri dish in an area about 2 cm in diameter. The embryos are maintained on this medium for 3-16 hour, preferably 4 hours, in complete darkness at 28° C. just prior to bombardment with particles associated with plasmid DNAs containing selectable marker and GFAT nucleic acids.

To effect particle bombardment of embryos, the particle-DNA agglomerates are accelerated using a DuPont PDS-1000 particle acceleration device. The particle-DNA agglomeration is briefly sonicated and 10 μl are deposited on macrocarriers and the ethanol is allowed to evaporate. The macrocarrier is accelerated onto a stainless-steel stopping screen by the rupture of a polymer diaphragm (rupture disk). Rupture is effected by pressurized helium. The velocity of particle-DNA acceleration is determined based on the rupture disk breaking pressure. Rupture disk pressures of 200 to 1800 psi can be used, with 650 to 1100 psi being preferred, and about 900 psi being most highly preferred. Multiple disks are used to effect a range of rupture pressures.

The shelf containing the plate with embryos is placed 5.1 cm below the bottom of the macrocarrier platform. To effect particle bombardment of cultured immature embryos, a rupture disk and a macrocarrier with dried particle-DNA agglomerates are installed in the device. The He pressure delivered to the device is adjusted to 200 psi above the rupture disk breaking pressure. A Petri dish with the target embryos is placed into the vacuum chamber and located in the projected path of accelerated particles. A vacuum is created in the chamber, preferably about 28 inches mercury. After operation of the device, the vacuum is released and the Petri dish is removed.

Bombarded embryos remain on the osmotically-adjusted medium during bombardment, and 1 to 4 days subsequently. The embryos are transferred to selection medium comprised of N6 basal salts, Eriksson vitamins, 0.5 mg/1 thiamine HCl, 30 gm/l sucrose, 1 mg/l 2,4-dichlorophenoxyacetic acid, 2 gm/l Gelrite, 0.85 mg/l silver nitrate, and 3 mg/l bialaphos (Herbiace, Meiji). Bialaphos was added filter-sterilized. The embryos are subcultured to fresh selection medium at 10 to 14 day intervals. After about 7 weeks, embryogenic tissue, putatively transformed for both selectable marker genes and GFAT nucleic acids, proliferate from about 7% of the bombarded embryos. Putative transgenic tissue is rescued, and that tissue derived from individual embryos is considered to be an event and was propagated independently on selection medium. Two cycles of clonal propagation are achieved by visual selection for the smallest contiguous fragments of organized embryogenic tissue.

A sample of tissue from each event is processed to recover DNA. The DNA is cleaved with a restriction endonuclease and probed with primer sequences designed to amplify DNA sequences overlapping the GFAT and non-GFAT portion of the plasmid. Embryogenic tissue with amplifiable sequence is advanced to plant regeneration.

For regeneration of transgenic plants, embryogenic tissue is subcultured to a medium comprising MS salts and vitamins (Murashige and Skoog, Physiol. Plant 15:473, 1962), 100 mg/l myo-inositol, 60 gm/l sucrose, 3 gm/l Gelrite, 0.5 mg/l zeatin, 1 mg/l indole-3-acetic acid, 26.4 ng/l cis-trans-abscissic acid, and 3 mg/l bialaphos in 100×25 mm Petri dishes, and the tissue is incubated in darkness at 28° C. until the development of well-formed, matured somatic embryos can be seen. This requires about 14 days. Well-formed somatic embryos are opaque and cream-colored, and are comprised of an identifiable scutellum and coleoptile. The embryos are individually subcultured to a germination medium comprising MS salts and vitamins, 100 mg/l myo-inositol, 40 gm/l sucrose and 1.5 gm/l Gelrite in 100×25 mm Petri dishes and incubated under a 16 hour light:8 hour dark photoperiod and 40 μeinsteinsm⁻²sec⁻¹ from cool-white fluorescent tubes. Typically, after about 7 days, the somatic embryos have germinated and have produced a well-defined shoot and root. The individual plants are subcultured to germination medium in 125×25 mm glass tubes to allow further plant development. The plants are maintained under a 16 hour light:8 hour dark photoperiod and 40 μeinsteinsm⁻²sec⁻¹ from cool-white fluorescent tubes. After about 7 days, the plants are well-established and are transplanted to horticultural soil, hardened off, and potted into commercial greenhouse soil mixture and grown to sexual maturity in a greenhouse. An elite inbred line is used as a male to pollinate regenerated transgenic plants.

(b) Use of GFAT Nucleic Acids to Modify the Characteristics of Plant Starch

Starch-producing plants that express an exogenous GFAT nucleic acid produce glucosamine-6-phosphate from glutamine and fructose-6-phosphate. Glucosamine-6-phosphate can be used to produce cationic starch by the following three steps. Endogenous phosphoglucomutase can convert glucosamine-6-phosphate to glucosamine-1-phosphate. This conversion can be enhanced by introducing an exogenous phosphoglucomutase nucleic acid into the plant. Suitable phosphoglucomutase nucleic acids include nucleic acids from Escherichia coli, Pseudomonas aeruginosa, Spinacia oleracea, Acetobacter xylinum, and rat liver (see, for example, Rivera et al., Gene 133:261, 1993, Brautaset et al., Microbiology 140:1183, 1994, Coyne et al., J. Bacteriol. 176:3500, 1994, Lu and Kleckner, J. Bacteriol 176:5847, 1994, and Penger et al., Plant Physiol. 105:1439, 1994).

UDP glucose pyrophosphorylase then converts glucosamine-1-phosphate to UDP glucosamine. The UDP glucose pyrophosphorylase may be endogenous or may be introduced into the plant with an exogenous UDP glucose pyrophosphorylase nucleic acid. Examples of suitable exogenous UDP glucose pyrophosphorylase nucleic acids include nucleic acids from Bacillus subtilis, Escherichia coli, Xanthomonas campestris, Saccharomyces cerevisiae, barley, bovine, and human liver (see, for example, Peng and Chang, FEBS Lett. 329:153, 1993, Soldo et al., J. Gen. Microbiol. 139:3185, 1993, Hossain et al., J. Biochem. 115:965, 1994, Daran et al., Eur. J. Biochem. 233:520, 1995, Eimert et al., Gene, 170:227, 1996, and Wei et al, Biochem. Biophys. Res. Commun. 226:607, 1996).

In the final step of cationic starch synthesis, glycogen synthase attaches UDP glucosamine moieties to starch molecules to produce starch comprising 2-amino anhydroglucose moieties. Suitable glycogen synthase nucleic acids include human liver and human muscle glycogen synthases (see, for example, Nuttall et al., Arch. Biochem. Biophys. 311:443, 1994, and Orho et al., Diabetes 44:1099, 1995).

Accordingly, a cationic starch-producing transgenic plant can be obtained using at least one expression vector encoding a GFAT, phosphoglucomutase, UDP glucose pyrophosphorylase, starch synthase, or glycogen synthase nucleic acid. All four exogenous nucleic acids need not be introduced into a plant if the plant has sufficient levels of phosphoglucomutase, UDP glucose pyrophosphorylase, and glycogen synthase activities. If it is desired to introduce GFAT and the remaining three nucleic acids into a plant, then one expression vector may include all four nucleic acids, or the four nucleic acids may be distributed among two, three or four expression vectors. Alternatively, one or more of the exogenous nucleic acids can be introduced into two inbred plants that are then crossbred to produce progeny with the desired traits.

The present invention, thus generally described, will be understood more readily by reference to the following examples, which are provided by way of illustration and are not intended to be limiting of the present invention.

### EXAMPLES

### Example 1

This example describes the construction of the cDNA libraries.

Total RNA Isolation

Total RNA was isolated from maize tissues with TRIzol Reagent (Life Technology Inc. Gaithersburg, Md.) using a modification of the guanidine isothiocyanate/acid-phenol procedure described by Chomczynski and Sacchi (Chomczynski, P., and Sacchi, N. Anal. Biochem. 162, 156 (1987)). In brief, plant tissue samples were pulverized in liquid nitrogen before the addition of the TRIzol Reagent, and then were further homogenized with a mortar and pestle. Addition of chloroform followed by centrifugation was conducted for separation of an aqueous phase and an organic phase. The total RNA was recovered by precipitation with isopropyl alcohol from the aqueous phase.

Poly(A)+ RNA Isolation

The selection of poly(A)+ RNA from total RNA was performed using PolyATact system (Promega Corporation. Madison, Wis.). In brief, biotinylated oligo(dT) primers were used to hybridize to the 3′ poly(A) tails on mRNA. The hybrids were captured using streptavidin coupled to paramagnetic particles and a magnetic separation stand. The mRNA was washed at high stringency conditions and eluted by RNase-free deionized water.

cDNA Library Construction

cDNA synthesis was performed and unidirectional cDNA libraries were constructed using the SuperScript Plasmid System (Life Technology Inc. Gaithersburg, Md.). The first strand of cDNA was synthesized by priming an oligo(dT) primer containing a Not I site. The reaction was catalyzed by SuperScript Reverse Transcriptase II at 45° C. The second strand of cDNA was labeled with alpha-32P-dCTP and a portion of the reaction was analyzed by agarose gel electrophoresis to determine cDNA sizes. cDNA molecules smaller than 500 base pairs and unligated adapters were removed by Sephacryl-S400 chromatography. The selected cDNA molecules were ligated into pSPORT1 vector in between of Not I and Sal I sites.

### Example 2

This example describes cDNA sequencing and library subtraction.

Sequencing Template Preparation

Individual colonies were picked and DNA was prepared either by PCR with M13 forward primers and M13 reverse primers, or by plasmid isolation. All the cDNA clones were sequenced using M13 reverse primers.

Q-bot Subtraction Procedure

cDNA libraries subjected to the subtraction procedure were plated out on 22×22 cm2 agar plate at density of about 3,000 colonies per plate. The plates were incubated in a 37° C. incubator for 12-24 hours. Colonies were picked into 384-well plates by a robot colony picker, Q-bot (GENETIX Limited). These plates were incubated overnight at 37° C.

Once sufficient colonies were picked, they were pinned onto 22×22 cm2 nylon membranes using Q-bot. Each membrane contained 9,216 colonies or 36,864 colonies. These membranes were placed onto agar plate with appropriate antibiotic. The plates were incubated at 37° C. for overnight.

After colonies were recovered on the second day, these filters were placed on filter paper prewetted with denaturing solution for four minutes, then were incubated on top of a boiling water bath for additional four minutes. The filters were then placed on filter paper prewetted with neutralizing solution for four minutes. After excess solution was removed by placing the filters on dry filter papers for one minute, the colony side of the filters were place into Proteinase K solution, incubated at 37° C. for 40-50 minutes. The filters were placed on dry filter papers to dry overnight. DNA was then cross-linked to nylon membrane by UV light treatment.

Colony hybridization was conducted as described by Sambrook, J., Fritsch, E. F. and Maniatis, T., (in Molecular Cloning: A laboratory Manual, 2nd Edition). The following probes were used in colony hybridization:

1. First strand cDNA from the same tissue as the library was made from to remove the most redundant clones.

2. 48-192 most redundant cDNA clones from the same library based on previous sequencing data.

3. 192 most redundant cDNA clones in the entire maize sequence database.

4. A Sal-A20 oligo nucleotide: TCG ACC CAC GCG TCC GM AAA AAA AAA AAA AAA AAA, removes clones containing a poly A tail but no cDNA.

5. cDNA clones derived from rRNA.

The image of the autoradiography was scanned into computer and the signal intensity and cold colony addresses of each colony was analyzed. Re-arraying of cold-colonies from 384 well plates to 96 well plates was conducted using Q-bot.

### Example 3

This example describes identification of the gene from a computer homology search. Gene identities were determined by conducting BLAST (Basic Local Alignment Search Tool; Altschul, S. F., et al., (1993) J. Mol. Biol. 215:403-410; see also www.ncbi.nlm.nih.gov/BLAST/) searches under default parameters for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences were analyzed for similarity to all publicly available DNA sequences contained in the “nr” database using the BLASTN algorithm. The DNA sequences were translated in all reading frames and compared for similarity to all publicly available protein sequences contained in the “nr” database using the BLASTX algorithm (Gish, W. and States, D. J. Nature Genetics 3:266-272 (1993)) provided by the NCBI. In some cases, the sequencing data from two or more clones containing overlapping segments of DNA were used to construct contiguous DNA sequences.

The above examples are provided to illustrate the invention but not to limit its scope. Other variants of the invention will be readily apparent to one of ordinary skill in the art and are encompassed by the appended claims. All publications, patents, patent applications, and computer programs cited herein are hereby incorporated by reference.

### Example 4

(a) Transformation of Hi-II Callus

Agrobacterium bacteria are streaked out from a −80° C. frozen aliquot onto a plate containing PHJ-L medium and cultured at 28° C. in the dark for 3 days. PHJ-L media comprises 25 ml/l Stock Solution A, 25 ml/l Stock Solution B, 450.9 ml/l Stock Solution C and spectinomycin (Sigma Chemicals, St. Louis, Mo.) is added to a concentration of 50 mg/l in sterile ddH₂O (stock solution A: K₂HPO₄ 60.0 g/l, NaH₂PO₄ 20.0 g/l, adjust pH to 7.0 with KOH and autoclave; stock solution B: NH₄Cl 20.0 g/l, MgSO₄.7H₂O 6.0 g/l, KCl 3.0 g/l, CaCl₂ 0.20 g/l, FeSO₄.7H₂O 50.0 mg/l, autoclave; stock solution C: glucose 5.56 g/l, agar 16.67 g/l (#A-7049, Sigma Chemicals, St. Louis, Mo.) and autoclave). A single colony is picked from the master plate and streaked onto a plate containing PHI-M medium [yeast extract (Difco) 5.0 g/l; peptone (Difco)10.0 g/l; NaCl 5.0 g/l; agar (Difco) 15.0 g/l; pH 6.8, containing 50 mg/L spectinomycin] and incubated at 28° C. in the dark for 2 days.

Five ml of either PHI-A, [CHU (N6) basal salts (Sigma C-1416) 4.0 g/l, Eriksson's vitamin mix (1000×, Sigma-1511) 1.0 ml/l; thiamine HCl 0.5 mg/l (Sigma); 2,4-dichlorophenoxyacetic acid (2,4-D, Sigma) 1.5 mg/l; L-proline (Sigma) 0.69 g/l; sucrose (Mallinckrodt) 68.5 g/l; glucose (Mallinckrodt) 36.0 g/l; pH to 5.2] for the PHI basic medium system, or PHI-I [MS salts (GIBCO BRL) 4.3 g/l; nicotinic acid (Sigma) 0.5 mg/l; pyridoxine HCl (Sigma) 0.5 mg/l; thiamine HCl 1.0 mg/l; myo-inositol (Sigma) 0.10 g/l; vitamin assay casamino acids (Difco Lab) 1.0 g/l; 2,4-D 1.5 mg/l; sucrose 68.50 g/l; glucose 36.0 g/l; adjust pH to 5.2 with KOH and filter-sterilize] for the PHI combined medium system and 5 μl of 100 mM 3′-5-dimethoxy-4′-hydroxyacetophenone (Aldrich Chemicals) are added to a 14 ml Falcon tube in a hood. About 3 full loops (5 mm loop size) Agrobacterium are collected from the plate and suspended in the tube, then the tube is vortexed to make an even suspension. One ml of the suspension is transferred to a spectrophotometer tube and the OD of the suspension is adjusted to 0.72 at 550 nm by adding either more Agrobacterium or more of the same suspension medium. The Agrobacterium concentration is approximately 1×10⁹ cfu/ml. The final Agrobacterium suspension is aliquoted into 2 ml microcentrifuge tubes, each containing 1 ml of the suspension. The suspensions are then used as soon as possible.

About two milliliters of the same medium (PHI-A or PHI-I) used for the Agrobacterium suspension are added into a 2 ml microcentrifuge tube. Immature embryos are isolated from a sterilized ear with a sterile spatula (Baxter Scientific Products S1565) and dropped directly into the medium in the tube. About 100 embryos are placed in the tube. The optimal size of the embryos is typically about 1.0-1.2 mm. The cap is then closed on the tube and the tube is vortexed with a Vortex Mixer (Baxter Scientific Products S8223-1) for 5 seconds at maximum speed. The medium is removed and 2 ml of fresh medium are added and the vortexing repeated. All of the medium is drawn off and 1 ml of Agrobacterium suspension is added to the embryos and the tube vortexed for 30 seconds. The tube is allowed to stand for 5 minutes in the hood. The suspension of Agrobacterium and embryos is poured into a Petri plate containing either PHI-B medium [CHU(N6) basal salts (Sigma C-1416) 4.0 g/l; Eriksson's vitamin mix (1000×, Sigma-1511) 1.0 ml/l; thiamine HCl 0.5 mg/l; 2.4-D 1.5 mg/l; L-proline 0.69 g/l; silver nitrate 0.85 mg/l; Gelrite (Sigma) 3.0 g/l; sucrose 30.0 g/l; acetosyringone 100 μM; pH 5.8], for the PHI basic medium system, or PHI-J medium [MS Salts 4.3 g/l; nicotinic acid 0.50 mg/l; pyridoxine HCl 0.50 mg/l; thiamine HCl 1.0 mg/l; myo-inositol 100.0 mg/l; 2,4-D 1.5 mg/l; sucrose 20.0 g/l; glucose 10.0 g/l; L-proline 0.70 g/l; MES (Sigma) 0.50 g/l; 8.0 g/l agar (Sigma A-7049, purified) and 100 μM acetosyringone with a final pH of 5.8] for the PHI combined medium system. Any embryos left in the tube are transferred to the plate using a sterile spatula. The Agrobacterium suspension is drawn off and the embryos placed axis side down on the media. The plate is sealed with Parafilm tape or Pylon Vegetative Combine Tape (E.G.CUT; Kyowa Ltd., Japan) and incubated in the dark at 23-25° C. for about 3 days of co-cultivation.

For the resting step, all of the embryos are transferred to a new plate containing PHI-C medium [CHU(N6) basal salts (Sigma C-1416) 4.0 g/l; Eriksson's vitamin mix (1000× Sigma-1511)1.0 ml/l; thiamine HCl 0.5 mg/l; 2,4-D 1.5 mg/l; L-proline 0.69 g/l; sucrose 30.0 g/l; MES buffer (Sigma) 0.5 g/l; agar (Sigma A-7049, purified) 8.0 g/l; silver nitrate 0.85 mg/l; carbenicillin 100 mg/l; pH 5.8]. The plate is sealed with Parafilm or Pylon tape and incubated in the dark at 28° C. for 3-5 days.

For selection, all of the embryos are then transferred from the PHI-C medium to new plates containing PHI-D medium, as a selection medium, [CHU (N6) basal salts (SIGMA C-1416) 4.0 g/l; Eriksson's vitamin mix (1000×, Sigma-1511) 1.0 ml/l; thiamine HCl 0.5 mg/l; 2,4-D 1.5 mg/l; L-proline 0.69 g/l; sucrose 30.0 g/l; MES buffer 0.5 g/l; agar (Sigma A-7049, purified) 8.0 g/l; silver nitrate 0.85 mg/l; carbenicillin (ICN, Costa Mesa, Calif.) 100 mg/l; bialaphos (Meiji Seika K. K., Tokyo, Japan) 1.5 mg/l for the first two weeks followed by 3 mg/l for the remainder of the time; pH 5.8] putting about 20 embryos onto each plate. The plates are sealed as described above and incubated in the dark at 28° C. for the first two weeks of selection. The embryos are transferred to fresh selection medium at two week intervals. The tissue is subcultured by transferring to fresh selection medium for a total of about 2 months. The herbicide-resistant calli are then “bulked up” by growing on the same medium for another two weeks until the diameter of the calli is typically about 1.5-2 cm.

For regeneration, the calli are then cultured on PHI-E medium [LMS salts 4.3 g/l; myo-inositol 0.1 g/l; nicotinic acid 0.5 mg/l, thiamine HCl 0.1 mg/l, pyridoxine HCl, 0.5 mg/l, glycine 2.0 mg/l, zeatin 0.5 mg/l, sucrose 60.0 g/l, agar (Sigma, A-7049) 8.0 g/l, indoleacetic acid (IAA, Sigma) 1.0 mg/l, abscisic acid (ABA, Sigma) 0.1 μM, Bialaphos 3 mg/l, carbenicillin 100 mg/l adjusted to pH 5.6] in the dark at 28° C. for 1-3 weeks to allow somatic embryos to mature. The calli are then cultured on PHI-F medium [MS salts 4.3 g/l; myo-inositol 0.1 g/l; thiamine HCl 0.1 mg/l, pyridoxine HCl 0.5 mg/l, glycine 2.0 mg/l, nicotinic acid 0.5 mg/l; sucrose 40.0 g/l; Gelrite 1.5 g/l; pH 5.6] at 25° C. under a daylight schedule of 16 hours light (270 uE m−²sec−¹) and 8 hours dark until shoots and roots developed. Each small plantlet is then transferred to a 25×150 mm tube containing PHI-F medium and grown under the same conditions for approximately another week. The plants are transplanted to pots with soil mixture in a greenhouse. Positive events are determined using methods similar to those used for examination of particle-bombarded transgenic maize at the callus stage or regenerated plant stage.

For Hi-II, a preferred optimized protocol typically includes 0.5×10⁹ cfu/ml Agrobacterium, a 3-5 day resting step, and no silver nitrate in the infection medium (PHI-A medium).

(b) Transformation of A188×Inbred Crosses

F₁ immature embryos are isolated from crosses of A188 to other inbreds and were subjected to transformation by Agrobacterium. The protocols are essentially the same as outlined above, with the following modifications. The Agrobacterium suspension is prepared with either the N6 salt containing medium, PHI-G [100 ml/l of a 10×solution of N6 macronutrients (463.0 mg/l (NH₄)₂SO₄, 400.0 mg/l KH₂PO₄, 125.33 mg/l CaCl₂, 90.37 mg/l MgSO₄ and 2,830.0 mg/l KNO₃), 2.44 mg/l boric acid, 37.1 mg/l Na₂-EDTA.2H₂O, 27.88 mg/l FeSO4.7H₂O, 7.33 mg/l MnSO₄.H₂O, 0.77 mg/l KI, 0.6 mg/l ZnSO₄.7H₂O, 0.15 mg/l Na₂MoO₂.2H₂O, 1.68 g/l KNO₃, 0.8 mg/l glycine, 3.2 mg/l nicotinic acid, 3.2 mg/l pyridoxine HCl, 3.4 mg/[thiamine HCl, 0.6 g/l Myo-inositol, 0.8 mg/l 2,4-D, 1.2 mg/l Dicamba (Sigma), 1.98 g/l L-proline, 0.3 g/l casein hydrolysate, 68.5 g/l sucrose and 36.0 g/l glucose, pH 5.2] or the MS salt-containing medium, PHI-I (supra) for the infection step. The co-cultivation medium is PHI-J (supra) and the co-cultivation time is about 3 to about 7 days. For PHJ90×A188, PHI-C medium (supra) is used in a 3 day resting step and PHI-D medium (supra) is used for selection. For PHN46×A188 and PHPP8×A188 transformations, no resting step is used, the co-cultivation time is about 5-7 days, and PHI-H medium [100 ml/l of a 10×solution of N6 macronutrients (463.0 mg/l (NH₄)₂SO₄, 400.0 mg/l KH₂PO₄, 125.33 mg/l CaCl₂, 90.37 mg/l MgSO₄ and 2,830.0 mg/l KNO₃), 2.44 mg/l boric acid, 37.1 mg/l Na₂-EDTA.2H₂O, 27.88 mg/l FeSO4.7H₂O, 7.33 mg/l MnSO₄.H₂O, 0.77 mg/l KI, 0.6 mg/l ZnSO₄.7H₂O, 0.15 mg/l Na₂MoO₂.2H₂O, 1.68 g/l KNO₃, 0.8 mg/l glycine, 3.2 mg/l nicotinic acid, 3.2 mg/l pyridoxine HCl, 3.4 mg/l thiamine HCl, 0.6 g/l Myo-inositol, 1.0 mg/l 2,4-D, 1.0 mg/l dicamba, 0.3 g/l casein hydrolysate, 20.0 g/l sucrose, 0.6 g/l glucose, 0.5 g/l MES buffer, 1 mg/l AgNO3, 5 mg/l bialaphos, 100 mg/l carbenicillin and 8.0 g/l Agar (Sigma A-7049, purified); pH 5.8] is used for selection. GFAT-positive events are determined at the callus stage or can be determined at the regenerated plant stage.

### Example 5

Evaluation of a Pathway for the Synthesis of Amino Starch in the Maize Endosperm

1. Biosynthetic Pathway for Producing Cationic Starch

A method for in vivo production of cationic starch (=amino starch) is outlined below. This requires a multi-step pathway, only part of which needs to be introduced through transgenes. A pathway to cationic starch is not normally found in maize or any other starch-storing plant tissue. A pathway for the synthesis can be constructed from known enzymatic activities. This requires a minimum of four enzymatic activities. The specifics are outlined below:

1. glutamine+fructose-6-phosphate→glutamate+glucosamine-6-phosphate

2. glucosamine-6-phosphate→glucosamine-1-phosphate

3. glucosamine-1-phosphate+UTP→UDP glucosamine+PPi

4. UDP glucosamine+starch→starch periodically interspersed with 2 amino anhydroglucose

These reactions are discussed, in turn, below.

Nucleic acids encoding glutamine:fructose-6-phosphate amidotransferase (GFAT), the enzyme for reaction 1 are characterized by SEQ ID NO: 1. This enzyme catalyzes the synthesis of glucose-6-phosphate with an amine group at position 2 of the glucosyl moiety. There is a rather extensive characterization of this enzyme in animal and fungal literature, but very little in that from plants.

GFAT production of glucosamine-6-phosphate is the first step in the new pathway leading to amino modified starch. Subsequent steps involve, the conversion of glucosamine-6-phosphate to glucosamine-1-phosphate by phosphoglucomutase (reaction 2). Utilization of glucosamine-1-phosphate to form the activated sugar nucleotide that serves as the hexosyl donor to the elongating starch molecule can come from the formation of UDP-glucosamine by UDP glucose pyrophosphorylase (UGPase) (reaction 3). The final step (reaction 4) involves incorporation of the glucosaminyl moiety of UDP glucosamine by starch or glycogen synthase(s).

GFAT has been cloned from a number of organisms and these sequences were used to BLAST our HGS database. We have isolated a full-length maize GFAT clone and maize GFAT transgenics have been generated for either endosperm-specific cytosolic or amyloplastidic expression by use of a seed-specific promoter.

We have demonstrated that maize phosphoglucomutase catalyzes the conversion of glucosamine-6-phosphate to glucosamine-1-phosphate. Using purchased yeast and bovine UGPase and maize endosperm extract, we showed the disappearance of glucosamine-1-phosphate and the appearance of a new peak in the HPLC display of the UGPase reaction products. Further characterization confirmed this new peak to be UDP glucosamine.

The normal substrate for maize starch synthases is ADP glucose, however, the granule bound starch synthase, waxy, is capable of utilizing UDP glucose as substrate. We have also isolated the yeast glycogen synthase and overexpressed it in Pichia. Preliminary results indicated that this enzyme was able to use UDP glucosamine to elongate glucosyl oligo primers.

Substrate specificity of these reactions was evaluated with alternative substrates that are required by the pathway leading to the synthesis of amino modified starch. The following sections present results from these experiments.

2. Activity Assay of GFAT Using Maize Endosperm Protein Extract

glutamine+fructose-6-phosphate→glutamate+glucosamine-6-phosphate

Reaction Components

60 mM KH₂PO₄, pH 7.0

1 mM EDTA

1 mM DTT

15 mM glutamine

15 mM fructose-6-Phosphate

Maize endosperm protein extract (200-μg total protein per 1.0-ml reaction)

Preparation of Endosperm Protein Extracts

Ground kernels (GS3, 20 days after pollination and mature kernels) in 60 mM KH₂PO₄, pH 7.0 with

protease inhibitors (protease inhibitor cocktail from Boehringer Mannheim).

Centrifuged at 25,000×g for 20 min at 4° C.

Used the supernatant as crude GFAT protein prep for the assay.

Incubate at 30° C. for 60 minutes.

Analyze by HPLC using Dionex's CarboPac-PA1 column with a pulsed amperometry detector (to detect formation of glucosamine-6-P):

| Time (min) | 100 mM NaOH | 100 mm NaOH/1 M sodium acetate |
|---|---|---|
| 0 | 90% | 10% |
| 20 | 80% | 20% |
| 30 | 70% | 30% |
| 50 | 50% | 50% |

Reactions with immature maize endosperm extract produced a prominent peak with a 23-minute retention and a minor one with a 28-minute retention, corresponding to the retention time for glucosamine-6-phosphate and fructose-6-phosphate, respectively. The large difference in peak area between the two peaks indicates the use of fructose-6-phosphate by GFAT in forming glucosamine-6-phosphate. In comparison, reactions with boiled immature endosperm extract used little fructose-6-phosphate and had little glucosamine-6-phosphate formed.

In similar reactions using endosperm extract prepared from dry, mature seeds, GFAT activity was found to be about 20% of that in immature endosperm.

Conclusions

Endogenous GFAT activity was detected in both immature and mature maize seeds, with the activity being significantly higher in immature seeds than in mature seeds. This HPLC-based GFAT assay protocol is used to analyze GFAT transgenic plants.

3. Activity Assay of Phosphoglucomutase (PGM)

glucosamine-6-phosphate→glucosamine-1-phosphate

Reaction Components:

80 mM triethanolamine, pH 7.6

2 mM EDTA

1 mM MgCl₂

20 μM glucosae-1,6-bisphosphate

2 mM glucosamine-1-phosphate or glucosamine-6-phosphate

Commercial PGM from rabbit muscle (Boehringer Mannheim, Catalog No. 108375)

Maize endosperm protein extract (200-μg total protein per 1.0 ml reaction)

Preparation of Maize Endosperm Protein Extract:

Ground kernels in ice-cold homogenization buffer (50 mM KH₂PO₄, pH 7.0; 5 mM EDTA; and 1 mM

DTT). Kernels were dissected from an ear (HN18) harvested 20 days after pollination.

Filtered the homogenate through 4-layer cheesecloth, and centrifuged at 25,000×g for 20 min at 4° C.

Desalted the supernatant into the homogenization buffer using a fast FPLC desalting column and collected the flow through.

Added PMSF and chymostatin to a final concentration of 1 mM and used the flow-through as crude PGM prep for the assay.

Incubated at 30° C. for 15 min.

* Transferred to a Centricon-30 concentrator and centrifuged at 1,400× for 20 min.

Removed 100 μl of the filtrate and added 400-μl water.

Injected 50 μl onto Dionex's CarboPac PA1 column and detected with a pulsed amperometry detector.

Eluted with 100 mM NaOH and 100 mM NaOH/1M sodium acetate (to detect formation of glucosamine-6-phosphate or glucosamine-1-phosphate):

| Time (min) | 100 mM NaOH | 100 mm NaOH/1 M sodium acetate |
|---|---|---|
| 0 | 90% | 10% |
| 20 | 80% | 20% |
| 30 | 70% | 30% |
| 50 | 50% | 50% |

Retention Time

| | | | |
|---|---|---|---|
| D-Glucose: | in void volume | D-Glucosamine: | in void volume |
| α-D-Glucose-1-P: | ˜12 min | α-D-Glucosamine-1-P: | ˜9 min |
| D-Glucose-6-P: | ˜25 min | D-Glucosamine-6-P: | ˜23.5 min |
| α-D-Glucose-1,6-DiP: | ˜32 min (Dionex) | | |

In reactions established as positive controls for the rabbit muscle PGM activity, the normal substrate glucose-1-phosphate was nearly depleted in forming glucose-6-phosphate, while no glucose-6-phosphate was detected in reactions with boiled rabbit muscle PGM. Reactions with the alternative substrate glucosamine-1-phosphate and the rabbit muscle PGM produced a 24-minute peak, corresponding to that of the glucosamine-6-phosphate standard. The conversion of glucosamine-1-phosphate to glucosamine-6-phosphate was estimated to be about 60% at the completion of reaction. No glucosamine-6-phosphate was detected in reactions with glucosamine-1-phosphate and boiled rabbit muscle PGM. Formation of glucosamine-1-phosphate was detected in reactions with glucosamine-6-phosphate as alternative substrate and rabbit muscle PGM and the conversion of glucosamine-6-phosphate to glucosamine-1-phosphate was about 70% at the end of reaction.

There was a near complete conversion of glucose-1-phosphate to glucose-6-phosphate in reactions with maize endosperm extract prepared from immature seeds. Glucosamine-6-phosphate was detected in similar reactions with glucosamine-1-phosphate as alternative substrate.

Conclusions

Commercial PGM was able to interconvert glucosamine-1-phosphate and glucosamine-6-phosphate. The studies using the commercial PGM also showed that the reaction favored the conversion of glucosamine-6-phosphate to glucosamine-1-phosphate. PGM activity (of converting glucosamine-1-phosphate to glucosamine-6-phosphate) was detected in maize endosperm prepared from seeds harvested 20 days after pollination.

4. Activity Assay of UDPglucose Pyrophosporylase (UGPase)

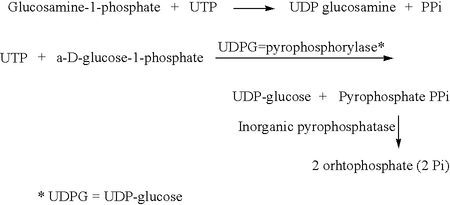

The above reaction is evaluated with an alternative substrate, glucosamine-1-phosphate.

Materials and Analysis of Reactions

Enzymes: UDP-glucose pyrophorylases purified from Bakers yeast (U 8501) & bovine liver (U 5877) by Sigma;

Inorganic pyrophosphatase from Boehringer Mannheim (Cat. No. 108 987)

Glucosamine-1-P, glucose-1-P from Sigma;

UDP, UMP, UTP, & UDP-glucose from Boehringer Mannheim;

UGPase assay was conducted per protocals used by Sigma;

For Baker Yeast UGPase

50 mM Tris-HCl, pH 7.6; 16 mM MgCl2; 10 mM L-cystein, 1 mM UTP; inorganic pyrophosphorylase; 2 mM glucose-1-P or glucosamine-1-P;

For Bovine Liver UGPase

48 mM Tris-HCl, pH 8.5; 19 mM MgCl₂; 2.3 mM β-mercaptoethanol; 1 mM UTP; inorganic pyrophosphorylase; 2 mM glucose-1-P or glucosamine-1-P;

For UGPase Activity in the Maize Endosperm

Preparation of Crude Maize Endosperm Protein Extract:

Weighted 8-g GS3 kernels (20 days after pollination);

Added 6-ml 100 mM Tris-HCl, pH 7.5 (11-22-96);

Added 0.5-ml Boehringer's protease inhibitor cocktail (one tablet dissolved in 1-ml water);

Blended for 10 sec, 3×at 4° C.;

Filtered through three layers of Cheesecloth at 4° C.;

Passed two 100-μl aliquots through a BioRad's BioSpin-30 column using a swing bucked rotor in Jouan at 2100 rpm, at RT, & repeated (i.e. two Bio-Spin-30 columns used for each of the 2, 100-μl aliquots);

Reaction Conditions

48 mM Tris-HCl, pH 8.5; 19 mM MgCl₂; 2.3 mM β-mercaptoethanol; 1 mM UTP; inorganic pyrophosphorylase; 2 mM glucose-1-P or glucosamine-1-P;

Carried out the reactions at 30° C. for 30 minutes;

Boiled for 5 min and passed through Centrion-10;

Analyzed the filtrate:

| | |
|---|---|
| System | HPLC anion exchange with Dionex's DX500/AS3500 |
| Column | Supelco's SAX1 (a strong anion exchange column), 25 cm × 4.6 |
| | mm ID, 5 μm particle; |

Elution Conditions:

| | |
|---|---|
| Buffer A | 10 mM acetic acid + 6 mM KH₂PO₄, pH 4.0; |
| Buffer B | 600 mM KH₂PO₄, pH 5.0; |

| Gradient Program: | Time (min) | % A | % B |
|---|---|---|---|
| | 0 | 100 | 0 |
| | 2 | 100 | 0 |
| | 40 | 81 | 19 |
| | 60 | 20 | 80 |
| | 65 | 20 | 80 |
| | 70 | 100 | 0 |

| Detection: | UV at 260 nm (Dionex AD20) (to detect formation of | | |
|---|---|---|---|
| | UDP glucose or UDP glucosamine); | | |
| Flow Rate: | 1.0 ml/min; | Temperature: | Ambient (˜23° C.); |
| Results | | | |

| | (min) | (min) |
|---|---|---|
| UMP | ˜12 | ˜13 |
| UDP-glucose | ˜24 | ˜25 |
| UDP | ˜27 | ˜27 |
| UTP | ˜41 | ˜39 |

The activity of the two commercial UGPases was confirmed by using glucose-1-phosphate as substrate. A large 24-minute peak was detected in reactions established as positive controls for the activity of both Baker yeast and bovine liver UGPases. This retention time is identical to that of UDP glucose standard. In comparison, little UDP glucose was formed in reactions with boiled either Baker yeast or bovine liver UGPase.

Using the alternative substrate glucosamine-1-phosphate, reactions with either Baker yeast or bovine liver UGPase produced a 10-minute peak that was absent in similar reactions with boiled enzyme prep. This unique putative UDP glucosamine peak was subject to further analysis described in the next section.

Similar reactivity in forming putative UDP glucosamine from glucosamine-1-phosphate and UTP was also detected in maize endosperm extract prepared immature seeds.

Conclusions

UGPase isolated from either Baker yeast or bovine liver recognized glucosamine-1-P as a substrate in the formation of UDP glucosamine. This activity in using glucosamine-1-P was also found in maize endosperm protein extract prepared from seeds harvested 20 days after pollination.

5. Purification by HPLC and Characterization by MALDI and ESI of Putative UDPglucosamine

UGPase reactions were carried out using bovine liver UGPase purchased from Sigma. The reactions were run using glucosamine1-phosphate and UTP as substrates and under the conditions described in the previous section.

The reactions were fractionated on Supecol's SAX1 HPLC column as described in the previous section. A 10-minute peak was eluted with approximately 8 mM acetic acid and 10 mM KH₂PO₄, pH 4.2. The peak was isolated which represented the UDPglucosamine.

An aliquot of this fraction was mixed at a ratio of 50:50 with a new mobile phase (acetonitrile:100 mM ammonium acetate; 75:25%) and loaded onto the same HPLC column. The chromatogram was developed with this new volatile buffer system using acetonitrile:100 mM ammonium acetate (75:25%). A ˜10-min peak of interest was collected. All fractions resulted from multiple runs were pooled and dried down using freeze-drier.

An aliquot of 1.0 mg of the dried fraction was analyzed with matrix-assisted laser desorption/ionization (MALDI) and electrospray ionization (ESI).

Results

Molecular Weight of UDP Glucosamine:

| | |
|---|---|
| Predicted based on structure | 565.3 dalton |
| Determined by MALDI | 565.4 dalton |
| Determined by ESI | 565.5 dalton |

Since the compound UDP glucosamine is not commercially available, further characterization was done to determine whether the unique 10-minute peak identified in the UGPase reaction indeed represented UDP glucosamine. Reactions conducted using glucosamine-1-phosphate and bovine liver UGPase were fractionated by HPLC and the unique 10-minute was isolated and analyzed by both MALDI and ESI. The molecular weight determined by either technique was essentially identical to that calculated on the basis of UDP glucosamine's structure. Therefore, it is concluded that the 10-minute peak produced in the UGPase reaction with glucosamine-1-phosphate as alternative substrate is UDP glucosamine.

Conclusions

The identity of the UDP glucosamine synthesized in vitro with bovine liver UGPase was confirmed by MALDI and ESI.

6. Activity Assay of the Yeast Glycogen Synthase (YGS)

The last reaction: synthesis of cationic starch by the yeast glycogen synthase overexpressed in Pichia using UDPgluosamine and glucosyl oligosaccharide primers:

UDP glucosamine+starch→starch periodically interspersed with 2 amino anhydroglucose

The YGS produced from secreted expression in Pichia was isolated from the growth medium, desalted into a buffer containing 50 mM Tris-HCl and 5 mM EDTA, pH 7.8, and concentrated using Centricon-30 concentrator. The preparation was used for assaying the activity of YGS.

Reaction Conditions:

50 mM Tris-HCl and 5 mM EDTA, pH 7.8

6.7 mM UDP glucose or UDP glucosamine

6.7 mM glucose-6-phosphate

1 mM maltoheptose (MW=153, from Sigma)

Protein prep: 150 μg/300-μl reaction

Incubated at 30° C. for 1 hour.

Analyze by HPLC using Dionex's CarboPac-PA1 column with a pulsed amperometry detector (to detect the formation of elongated glusyl oligo primers);

| Time (min) | 100 mM NaOH | 150 mm NaOH/500 mM sodium acetate |
|---|---|---|
| 0 | 100% | 0% |
| 50 | 0% | 100% |

YGS overexpressed in Pichia was assayed for its use of UDP glucosamine in elongation of oligosaccharide primers. A 28-minute peak was detected in reactions with the crude protein prep and not detected in reactions with the boiled protein prep. This peak had a retention time longer than that for any of the maltoheptose primer peaks. Since under the elution conditions used in this study, larger oligosaccharides have longer retention on the column, the results suggest that YGS was able to recognize UDP glucosamine in synthesis of amino starch.

Conclusions

Preliminary results from this study suggest that the yeast glycogen synthase recognize UDP glucosamine as a substrate and transfer the glucosaminyl moiety to glucosyl oligo primers.

7. Analysis of GFAT Transgenic Plants (Two Constructs)

Two GFAT constructs were built for maize transformation:

Construct 12405: gamma zein promoter::GFAT::gamma zein and

Construct 12413: gamma zein promoter:bt1 transient peptide::GFAT::gamma zein.

T1 seeds from GFAT transformants were analyzed for expression of GFAT protein and accumulation of amino starch in the endosperm as well as intermediate metabolites involved in the pathway. Expression of GFAT protein was detected with Western blot in T1 seeds transformed with either constructs and no endogenous GFAT was detected in non-transformed GS3 seeds.

Endosperm meal from the GFAT transgenic plants was extensively washed in water to remove free sugars, and hydrolyzed by boiling for 3 hours in 100 mM HCl. Following centrifugation, the supernatant was passed through a Microcon-3 concentrator and the filtrate was analyzed with HPLC to detect the presence of glucosamine, glucosamine1-phosphate, glucosamine-6-phosphate, and/or UDPglucosamine.

No glucosamine was detected in the samples, suggesting there was no amino starch synthesized. It seems that the starch synthase cannot recognize UDP glucosamine as substrate. One Construct 12405 event was found to have an elevated level of glucosamine-1-P and several Construct 12413 events showed HPLC-detectable level of UDP glucosamine accumulation. Future experiments will employ yeast glycogen synthase to produce cationic starch.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

What is claimed is:

1. An isolated nucleic acid comprising a polynucleotide encoding a full-length maize glutamine:fructose-6-phosphate amidotransferase (GFAT) polypeptide or a complement of the polynucleotide.

2. An isolated nucleic acid comprising the sequence set forth in SEQ ID NO: 1 or a complement thereof.

3. An isolated nucleic acid comprising a polynucleotide that encodes a polypeptide of SEQ ID NO 2.

4. The isolated nucleic acid molecule of claim 1, wherein the polypeptide is a functional fragment of the polypeptide of SEQ ID NO: 2.

5. An expression cassette comprising the nucleic acid of claim 3 operably linked to a promoter, wherein the nucleic acid is in sense or antisense orientation.

6. A recombinant plant host cell comprising the expression cassette of claim 5.

7. The recombinant host cell of claim 6 wherein the cell is obtained from a plant selected from the group consisting of: maize, sorghum, wheat, rice, barley, oats, sunflower, soybean, Brassica, cassava, sweet potato, and potato.

8. The recombinant host cell of claim 7, wherein the cell is a maize cell.

9. A plant regenerated from the host cell of claim 6.

10. A plant according to claim 9 wherein the host cell is from maize.

11. A method for increasing the level of UDP-glucosamine in a plant, comprising:
(a) stably transforming a plant cell with the polynucleotide of claim 3, operably linked to a promoter; and
(b) regenerating a plant from the recombinant plant cell, to produce a plant capable of expressing the polynucleotide for a time sufficient to increase the level of UDP-glucosamine in the plant.


SEQUENCE LISTING (4 sequences)

SEQ ID NO: 1 - DNA, 2428 bp, Zea mays
gtcttcgccgctcccttcccggcctcccgggctggacgaaacgaaccctcgctcgccctccttataaccgaacggccgaacccagccaacccagccgtttctcttcgtacggcctctgccagccagtgtcctgctactagggaagcataccaactccccattcttctcttcgccgcagccaggaaggaaggatgtgcgggatcttcgcctacctcaactacaacgtctcgcgggagcgccgctacatcctcgaggtcctcttcaacggcctccgccgcctcgagtaccgcggctacgactccgccgggatcgcgctcgatgccgaccgccaggtcccctcccccgctcccgcttcctcttccgacgcgcggccgtacgccggggcgccgccgctcgtgttccgccaggagggcaagatcgagaacctcgtgcgatccgtctactccgaggttgatgagaaggatgtgaacctggatgctgcgttcagtgtgcatgctgggatcgcacataccaggtgggccacgcacggtgtgcctgctccaaggaacagccacccccaatcgtctggtgccggtgatgagttcttggttgtccacaatggcattatcaccaactatgaggtcttgaaagagacactaactaggcacggcttcacctttgagtctgatacagacacagaagtcatccctaagctagcaaagttcgtttttgataaatctcatgatgaacaaggtgatgtgacgtttagccaagttgttatggaagtcatgaggcagcttgaaggagcctacgcacttatctttaaaagcccgcactatcccaatgaattgattgcatgcaaacgaggcagccaactgatacttggtgtcaacgaattgagtggtcaacagaatgggaaatcatttcatgatgtcaaaaccttgacaacaaatggaaagcccaaagaattattcttctccagtgatctatgtgctattgtagagcatacgaagaactacttagctcttgaagataatgaaattgttcatattaaggatggtagtgtttcgatcctcaagtttgaccctcacaaagagaagccagcatctgtgcaacgagcattgtctgttcttgagatggaagttgagcaaataaagaaaggaagttatgatcacttcatgcaaaaagaaatccatgaacagccacattcgttgaaaacaacaatgaggggtagattgaaggatggtggggttgttctaggtggactgaaggaatatctcaagacaattaggcgctgtagaagggtggtatttattggttgtggaacaagttacaatgctgccttagctgcaagaccttttgtggaagaactgactggtattcctgtgactatggaggttgcaagtgacttgctggacagacaaggtcccatctacagagaagacactgcagtttttgttagtcaatctggggagacagcagataccctccttgctctagattatgcactagaaaatggagctctctgtgttggcataacaaatactgttggaagcacgctgtctagaaaaacacactgtggggttcatatcaatgctggttgtgagattggtgttgccagtacaaaggcttatacaagtcaaatagtagccatggcgatgatggcgttggctattgggtccgatcagatatctactcaagctaggagggacagtatcatcagtggactgaacaacctttcaagcaatgtcagcgaagttctcaagctagatgctggaatgaaggagcttgcctcttcgctgatcgactcagagtcgctcctcgtgttcggaaggggttacaactacgccaccgcgctggagggcgccctgaaggtcaaggaggtggcgctgatgcacagcgagggcatgctcgctggcgagatgaagcacgggccgctggccctcgtggacgagaacctccccatcattgtcattgcgacccgcgacgcgtgcttcagcaagcagcagtcggtgatccaacagctcctctcgcgcagggggcgcctgatagtgatgtgctctaggggagatgccgcggctgtgtgccctagcggtgggtcgtgcagagtcattgaagttccacaggttgcagactgtctccagccagtgatcaacataattccattacagttgctcgcgtaccatctgactgttctccggggattcgacgtggaccaaccaaggaatctggcgaagagcgtgaccacgcagtagggagaggtagatgagatgtttgtattgtagttaattgtccttgctcttgaggtggctagtacgtagcataaatattatggtgcgttaaacttgttgttttgtgaacgaaatgtacctctctttttttaattatggtatattggtgtcaatagcaaaaaaaaaaaaaaaaaaaaaaaaaaaaaaaaaaaaaaaaa

SEQ ID NO: 2 - protein, 680 aa, Zea mays
MCGIFAYLNYNVSRERRYILEVLFNGLRRLEYRGYDSAGIALDADRQVPSPAPASSSDARPYAGAPPLVFRQEGKIENLVRSVYSEVDEKDVNLDAAFSVHAGIAHTRWATHGVPAPRNSHPQSSGAGDEFLVVHNGIITNYEVLKETLTRHGFTFESDTDTEVIPKLAKFVFDKSHDEQGDVTFSQVVMEVMRQLEGAYALIFKSPHYPNELIACKRGSQLILGVNELSGQQNGKSFHDVKTLTTNGKPKELFFSSDLCAIVEHTKNYLALEDNEIVHIKDGSVSILKFDPHKEKPASVQRALSVLEMEVEQIKKGSYDHFMQKEIHEQPHSLKTTMRGRLKDGGVVLGGLKEYLKTIRRCRRVVFIGCGTSYNAALAARPFVEELTGIPVTMEVASDLLDRQGPIYREDTAVFVSQSGETADTLLALDYALENGALCVGITNTVGSTLSRKTHCGVHINAGCEIGVASTKAYTSQIVAMAMMALAIGSDQISTQARRDSIISGLNNLSSNVSEVLKLDAGMKELASSLIDSESLLVFGRGYNYATALEGALKVKEVALMHSEGMLAGEMKHGPLALVDENLPIIVIATRDACFSKQQSVIQQLLSRRGRLIVMCSRGDAAAVCPSGGSCRVIEVPQVADCLQPVINIIPLQLLAYHLTVLRGFDVDQPRNLAKSVTTQ

SEQ ID NO: 3 - DNA, 237 bp, Zea mays
atggcggcgacaatggcagtgacgacgatggtgaccaggagcaaggagagctggtcgtcattgcaggtcccggcggtggcattcccttggaagccacgaggtggcaagaccggcggcctcgagttccctcgccgggcgatgttcgccagcgtcggcctcaacgtgtgcccgggcgtcccggcggggcgcgacccgcgggagcccgatcccaaggtcgtccgggcggccgacctcatg

SEQ ID NO: 4 - DNA, 36 bp, Artificial Sequence
  SalA-20 oligo
tcgacccacgcgtccgaaaaaaaaaaaaaaaaaaaa